(19) United States
(12) Patent Application Publication
(10) Pub. No.: US 2005/0100547 A1
(43) Pub. Date: May 12, 2005

(54) Sphingosine kinase interacts with traf2 and modulates tumor necrosis factor-induced cellular activity

(75) Inventors: Pu Xia, Magill (AU); Lijun Wang, Magill (AU); Mathew Vadas, Stirling (AU); Jennifer Gamble, Stirling (AU); Paul Moretti, Goodwood (AU); Stuart Pitson, Norwood (AU)

Correspondence Address: SEED INTELLECTUAL PROPERTY LAW GROUP PLLC, 701 FIFTH AVE, SUITE 6300, SEATTLE, WA, 98104-7092, US

(73) Assignee: Pu XIA etal

(21) Appl. No.: 10/479,933

(22) PCT Filed: Jun. 3, 2002
(86) PCT No.: PCT/AU02/00710

(30) Foreign Application Priority Data
Jun. 7, 2001 (AU) PR 5521
Aug. 13, 2001 (AU) PR 6978
Dec. 27, 2001 (AU) PR 9759

(51) Int. Cl. A61K 39/395; A61K 38/19; A61K 38/45

(52) U.S. Cl. 424/144.1; 514/12; 424/85.1

(57) ABSTRACT

The present invention relates generally to a method of modulating cytokine-mediated cellular activity and to agents useful for same. More particularly, the present invention contemplates a method of modulating tumor necrosis factor-mediated cellular activity by modulating an intracellular sphingosine kinase-dependent signalling mechanism. The method of the present invention is useful, inter alia, in the treatment and/or prophylaxis of conditions characterised by aberrant, unwanted or otherwise inappropriate cytokine-mediated cellular activity The present invention is further directed to methods for identifying and/or designing agents capable of modulating the subject sphingosine kinase-dependent signalling mechanism.

### FIELD OF THE INVENTION

The present invention relates generally to a method of modulating cytokine-mediated cellular activity and to agents useful for same. More particularly, the present invention contemplates a method of modulating tumor necrosis factor-mediated cellular activity by modulating an intracellular sphingosine kinase-dependent signalling mechanism. The method of the present invention is useful, inter alia, in the treatment and/or prophylaxis of conditions characterised by aberrant, unwanted or otherwise inappropriate cytokine-mediated cellular activity The present invention is further directed to methods for identifying and/or designing agents capable of modulating the subject sphingosine kinase-dependent signalling mechanism.

### BACKGROUND OF THE INVENTION

Bibliographic details of the publications referred to by author in this specification are collected at the end of the description.

The reference to any prior art in this specification is not, and should not be taken as, an acknowledgment or any form of suggestion that that prior art forms part of the common general knowledge in Australia.

Tumor necrosis factor-α (herein referred to as “TNF”), originally defined by its tumoricidal activity, is a pleiotropic cytokine which elicits a wide spectrum of organismal and cellular responses such as cell proliferation, apoptosis, and inflammatory and immunoregulatory responses. The different cellular responses to TNF are signalled through cell surface receptors (p55 TNF-R1 and p75 TNF-R2), and their adaptor proteins, initiating distinct and separate signalling pathways. These separate signals can lead to opposing cellular effects as best exemplified by TNF's both apoptotic and anti-apoptotic role (Locksley et al., Cell 104, 487, 2001).

The strikingly different cellular responses to tumor necrosis factor, such as cell survival, activation and apoptosis, are signalled through the separate pathways. Discrete signalling is believed to be initiated by recruiting difference types of adaptor proteins to the TNF receptor superfamily complexes. For example, the recruitment of a complex including TRADD, FADD/MORT1 and RIP leads to the further recruitment and activation of various caspases and, subsequently, to cell death (Tartaglia et al., Cell 73, 213, 1993; Chinnaiyan et al., Science 274, 990, 1996). On the other hand, TNF induces the interaction of its receptor with a second class of adaptor protein, TNFR-associated factors (herein referred to as “TRAFs”) and recruits downstream signals such as NF-κB-inducing kinase (NIK) to activate NF-κB (Locksley et al., 2001 supra; Arch et al., Genes & Devel. 12, 2821, 1998). In addition, the recruitment of TRADD, FADD and FAN to TNF receptors enables activation of either acidic or neutral sphingomyelinase (Smase) yielding ceramide that has been implicated in the apoptotic signalling in several cell types. TNF activates another key enzyme, sphingosine kinase (SphK), in the sphingomyelin metabolic pathway resulting in production of sphingosine-1-phosphate (S1P). S1P is a potent antagonist of TNF mediated apoptosis.

The TNF receptor-associated factors comprise six members, being TRAF1-6. TRAF2 is the prototypical member of the TRAF family. It can interact directly or indirectly with a number of TNF receptors to mediate the signal transduction of these receptors. TRAF2 can also interact with numerous intracellular proteins such as I-TRAF/TANK, RIP, NIK and the caspase inhibitors cIAPs, and thereby transduces signals required for the activation of the transcription factor NF-κB, the stress-activated protein kinase (SAPK or JNK) and anti-apoptosis (Hsu et al., Cell 81, 495, 1995; Rothe et al. Cell 83, 1243, 1995; Wang et al, Science 281, 1680, 1998). Structural studies have revealed the complexity and flexibility of TRAF2 as a signal junction (Park et al., Cell 101, 777, 2000). However, it is still not clear whether TRAF2 can differentially activate distinct downstream signals such as NF-κB and JNK, leading to different biological functions or how this may be achieved. Indeed, targeted gene deletion or truncated TRAF2 (ΔTRAF2) which lacks the N-terminal 86 amino acids that comprise the RING finger domain has showed that TRAF2 is essential for JNK activation and the suppression of TNF-induced apoptosis via NF-(B-independent pathways) (Yeh et al., Immunity 7:715, 1997; Lee et al., Immunity 7:703, 1997), but the precise mechanism of this function has not been established.

Sphingolipids have recently emerged as signal molecules to mediate the pleiotropic activities of TNF (Hannun et al., Biochemistry 40:4893, 2001). The TNF signalling via sphingolipid turnover is exemplified by two distinct pathways: the formation of ceramide resulting from the activation of sphingomyelinase (SMase) and the production of sphingosine-1-phosphate (S1P) upon sphingosine kinase (SphK) activation. Despite the controversy regarding ceramide as an apoptotic inducer to mediate TNF-induced cell death (Hannun, Science 274:1855, 1996), S1P has been evident as an anti-apoptotic and mitogenic factor (Olivera and Spiegal, Nature 365:557 1993; Cuvillier et al., Nature 381:800, 1996; Xia et al., J. Biol. Chem. 27434499, 1999 and Cur. Biol. 10:1527, 2000).

The signals which regulate TNF-mediated cellular activity have not been fully defined. Elucidation of these cellular signalling mechanisms is necessary for the development of therapeutic and/or prophylactic strategies directed to treating conditions characterised by aberrant or otherwise unwanted cellular activity, which cellular activity is directly or indirectly modulatable via a TNF-regulated signalling mechanism. Whilst structural studies have revealed the complexity and flexibility of TRAF2 (Park et al., 2000, supra) as a signal junction, it is still not clear whether and how TRAF2 can differentially activate its distinct downstream signals such as NF-κB and JNK, leading to different biological functions.

In work leading up to the present invention, the inventors have identified a physical interaction between TRAF2 and sphingosine kinase (SphK), a lipid kinase that is responsible for the production of sphingosine-1-phosphate (S1P). The interaction of TRAF2 and SphK is capable of activating SphK, which is required for TRAF2 mediated activation of NF-(B but not JNK. In addition, it has been determined that TRAF2-promoted anti-apoptotic signal pathways critically involves SphK activation during TNF-mediated apoptosis. Accordingly, the interaction between the TNF receptor superfamily and sphingolipid signal pathways provides a mechanism of TRAF2-mediated signals specifically leading to cell activation and survival. The inventors have still further identified the TRAF2 binding motif of sphingosine kinase. The identification of these cellular signalling mechanisms and binding motif has now facilitated the development of methodology directed to modulating TNF mediated cellular activity by regulating the interaction of sphingosine kinase with TRAF. Identification of the sphingosine kinase/TRAF2 binding motif has still further facilitated the development of methodology directed to the identification, design and use of agents which interact with this binding motif thereby modulating TNF mediated cellular activity.

### SUMMARY OF THE INVENTION

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

As used herein, the term “derived from” shall be taken to indicate that a specified integer may be obtained from a particular source albeit not necessarily directly from that source.

The subject specification contains amino acid sequence information prepared using the programme PatentIn Version 3.1, presented herein after the bibliography. Each amino acid sequence is identified in the sequence listing by the numeric indicator <210> followed by the sequence identifier (e.g. <210>1, <210>2, etc). The length, type of sequence (protein (PRT), etc) and source organism for each amino acid sequence are indicated by information provided in the numeric indicator fields <211>, <212> and <213>, respectively. Amino acid sequences referred to in the specification are defined by the information provided in numeric indicator field <400> followed by the sequence identifier (eg. <400>1, <400>2, etc).

One aspect of the present invention provides a method of modulating cytokine-induced cellular activity, said method comprising contacting said cell with an effective amount of an agent for a time and under conditions sufficient to modulate the interaction of sphingosine kinase with a TRAF wherein inducing or otherwise agonising said association up-regulates said cellular activity and inhibiting or otherwise antagonising said association down-regulates said cellular activity.

In another aspect the present invention provides a method of modulating TNF-induced cellular activity, said method comprising contacting said cell with an effective amount of an agent for a time and under conditions sufficient to modulate the interaction of sphingosine kinase with a TRAF wherein inducing or otherwise agonising said association up-regulates said cellular activity and inhibiting or otherwise antagonising said association down-regulates said cellular activity.

Yet another aspect of the present invention provides a method of modulating TNF induced cellular activity said method comprising contacting said cell with an effective amount of an agent for a time and under conditions sufficient to modulate the interaction of sphingosine kinase with TRAF2 wherein inducing or otherwise agonising said association up-regulates said cellular activity and inhibiting or otherwise antagonising said association down-regulates said cellular activity.

Still another aspect of the present invention provides a method of down-regulating the TNF induced anti-apoptotic functional activity of a cell, said method comprising contacting said cell with an effective amount of an agent for a time and under conditions sufficient to down-regulate the interaction of sphingosine kinase with TRAF2 wherein inhibiting or otherwise antagonising said interaction up-regulates the apoptosis of said cell.

In another aspect there is provided a method of down-regulating the TNF-induced pro-inflammatory functional activity of a cell, said method comprising Contacting said cell with an agent for a time and under conditions sufficient to down regulate the interaction of sphingosine kinase with TRAF2 wherein inhibiting or otherwise antagonising said interaction down-regulates one or more of said cells TNF induced pro-inflammatory activities.

In still another aspect the present invention provides a method of modulating cytokine induced cellular activity said method comprising contacting said cell with an effective amount of an agent for a time and under conditions sufficient to modulate the interaction of sphingosine kinase with a TRAF, which agent binds, links or otherwise associates with the C-terminal region of sphingosine kinase, wherein inducing or otherwise agonising said interaction up-regulates said cellular activity and inhibiting or otherwise antagonising said interaction down-regulates said cellular activity.

In yet still another aspect the present invention provides a method of modulating TNF induced cellular activity said method comprising contacting said cell with an effective amount of an agent for a time and under conditions sufficient to modulate the interaction of sphingosine kinase with TRAF2, which agent binds, links or otherwise associates with the C-terminal region of sphingosine kinase, wherein inducing or otherwise agonising said interaction up-regulates said cellular activity and inhibiting or otherwise antagonising said interaction down-regulates said cellular activity.

A further aspect of the present invention provides a method of modulating TNF induced cellular activity said method comprising contacting said cell with an effective amount of an agent for a time and under conditions sufficient to modulate the interaction of sphingosine kinase with TRAF2, which agent binds, links or otherwise associates with the amino acid sequence PPEE at the C-terminal region of sphingosine kinase, wherein inducing or otherwise agonising said interaction up-regulates said cellular activity and inhibiting or otherwise antagonising said interaction down-regulates said cellular activity.

Still another aspect of the present invention is directed to the method for the treatment and/or prophylaxis of a conditions characterised by aberrant, unwanted or otherwise inappropriate cytokine induced cellular activity in a mammal, said method comprising administering to said mammal an effective amount of an agent for a time and under conditions sufficient to modulate the interaction of sphingosine kinase with a TRAF wherein inducing or otherwise agonising said interaction up-regulates said cellular activity and inhibiting or otherwise antagonising said interaction down-regulates said cellular activity.

Yet still a further aspect of the present invention provides a method for the treatment and/or prophylaxis of a condition characterised by aberrant, unwanted or otherwise inappropriate TNF induced cellular activity in a mammal, said method comprising administering to said mammal an effective amount of an agent for a time and under conditions sufficient to modulate the interaction of sphingosine kinase with a TRAF wherein inducing or otherwise agonising said interaction up-regulates said cellular activity and inhibiting or otherwise antagonising said interaction down-regulates said cellular activity.

In another aspect there is provided a method for the treatment and/or prophylaxis of a neoplastic condition said method comprising administering to said mammal an effective amount of an agent for a time and under conditions sufficient to down-regulate the interaction of sphingosine kinase with TRAF2 wherein inhibiting or otherwise antagonising said interaction down-regulates TNF induced anti-apoptotic functional characteristics.

In still another aspect there is provided a method for the treatment and/or prophylaxis of inflammation in a mammal, said method comprising administering to said mammal an effective amount of an agent for a time and under conditions sufficient to down-regulate the interaction of sphingosine kinase with TRAF2 wherein inhibiting or otherwise antagonising said interaction down-regulates the production of TNF induced inflammatory mediators by said mammal.

Yet another aspect of the present invention contemplates the use of an agent, as hereinbefore defined, in the manufacture of medicament for the treatment of a condition in a mammal, which condition is characterised by aberrant, unwanted or otherwise inappropriate cytokine induced cellular activity, wherein said agent modulates the interaction of sphingosine kinase with a TRAF and wherein inducing or otherwise agonising said interaction up-regulates said cellular activity and inhibiting or otherwise antagonising said interaction down-regulates said cellular activity.

In yet another further aspect, the present invention contemplates a pharmaceutical composition comprising the modulatory agent as hereinbefore defined together with one or more pharmaceutically acceptable carriers and/or diluents. Said agents are referred to as the active ingredients.

Yet another aspect of the present invention relates to the agent as hereinbefore defined, when used in the method of the present invention.

Another aspect of the present invention provides a method for detecting an agent capable of modulating the interaction of TRAF with sphingosine kinase or its functional equivalent or derivative thereof said method comprising contacting a cell or extract thereof containing said sphingosine kinase and TRAF or its functional equivalent or derivative with a putative agent and detecting an altered expression phenotype associated with said interaction.

A further aspect of the present invention provides a method for detecting an agent capable of modulating the interaction of TRAF with sphingosine kinase or its functional equivalent or derivative thereof said method comprising contacting a cell containing said sphingosine kinase and TRAF or its functional equivalent or derivative with a putative agent and detecting an altered apoptosis profile associated with said interaction.

In yet another aspect the present invention provides a method for detecting an agent capable of binding or otherwise associating with the TRAF2 binding site of sphingosine kinase or functional equivalent or derivative thereof said method comprising contacting a cell containing said binding site or functional equivalent or derivative thereof with a putative agent and detecting an altered expression phenotype associated with modulation of the function of sphingosine kinase or its functional equivalent or derivative.

Still yet a further aspect of the present invention is directed to a method for analysing, designing and/or modifying an agent capable of interacting with the TRAF binding site of sphingosine kinase or derivative thereof and modulating at least one functional activity associated with said sphingosine kinase said method comprising contacting said sphingosine kinase or derivative thereof with a putative agent and assessing the degree of interactive complementarity of said agent with said binding site.

In a related aspect, the present invention should be understood to extend to the agents identified utilising any of the methods hereinbefore defined. In this regard, reference to an agent should be understood as a reference to any proteinaceous or non-proteinaceous molecule which modulates at least one TRAF mediated functional activity.

Single and three letter abbreviations used throughout the specification are defined in Table 1.

TABLE 1
Single and three letter amino acid abbreviations

| | Three-letter | One-letter |
|---|---|---|
| Amino Acid | Abbreviation | Symbol |
| Alanine | Ala | A |
| Arginine | Arg | R |
| Asparagine | Asn | N |
| Aspartic acid | Asp | D |
| Cysteine | Cys | C |
| Glutamine | Gln | Q |
| Glutamic acid | Glu | E |
| Glycine | Gly | G |
| Histidine | His | H |
| Isoleucine | Ile | I |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |
| Any residue | Xaa | X |

### BRIEF DESCRIPTION OF THE DRAWINGS

FIG. 1 is an image of the effect of TRAF2 on SphK activation. 293T cells were transfected with TRAF2, ΔTRAF2, or empty expression vectors. 48 h post-transfection, cells were stimulated with or without TNF (1 ng/ml) for 10 min, and cell lysates were prepared. (a) SphK activity was measured in the cytosolic fractions as described under Methods. Data are the mean (±S.D.) of three individual experiments and each experiment was done in duplicate. (b) Immunoblotting assays with anti-FLAG antibodies showed equivalent expression of the transgenes.

FIG. 2 is an image of the Interaction of TRAF2 and SphK. 293T cells were cotransfected with the indicated amounts (μg) of expression vectors. 48 h after transfection and where indicated stimulation with TNF (1 ng/ml) for 10 min, whole-cell lysates were prepared. (a) The lysates were immunoprecipitated (IP) with anti-FLAG or mouse control IgG antibodies as indicated. The IP complexes were then analysed by immunoblotting assay (IB) with anti-FLAG (top) or anti-HA antibodies (bottom). (b) The lysates from HUVEC or transfected 293T cells were incubated with GST-SphK (lane 7-9) or GST alone (lane 4-6) fusion proteins. Proteins coprecipitated with GST fusion proteins, along with whole lysates (lane 1-3), were analyzed by an immunoblot using anti-TRAF2 antibodies.

FIG. 3 is an image depicting that the site-directed mutagenesis of TRAF2-binding motif in SphK ablates the interaction of SphK with TRAF2. (a) Diagrams of the putative TRAF2-binding motifs (TB1 and TB2) in wild-type human SphK-1 (wt-SphK) and the mutants of SphK, TB1-SphK, TB2-SphK and TB1/2-SphK. (b) 293T cells were cotransfected with the indicated expression vectors. 48 h after transfection, cells were lysed and the lysates were immunoprecipitated (IP) with anti-TRAF2 antibodies and coimmunoprecipitated SphK or its mutants were detected by immunoblotting assay (IB) with anti-FLAG antibodies (top panel). The expression of proteins in whole-cell lysates was shown in bottom panel.

FIG. 4 is a graphical representation of TB2-SphK and SphK^{G82D} blocking TNF-induced SphK activation. (a) 293T cells were transfected with the indicated expression vectors, and SphK activity was determined after stimulation with TNF (1 ng/ml), PMA (100 nM) or nil for 10 min post-transfection 48 h. Data are the mean (±S.D.) of relative activity of three individual experiments. (b) SphK activity was assayed in the SphK- or TB2-SphK-transfected 293T cells at the indicated time points of TNF (1 ng/ml) stimulation. Data shown are mean of activity of one representative experiment done in duplicate.

FIG. 5 is an image of the effect of SphK on NF-κB activation. 293T cells were cotransfected the indicated expression vectors. 48 h after the transfection, cells were stimulated with or without TNF (1 ng/ml) for 30 min. (a) Western blot assay with anti-IκBα antibodies showing I-κBα degradation. (b) NF-κB activation was determined by gel shift assay of NF-κB DNA binding complex as described in the Methods. * Individual reactions were supplemented with a 50-fold excess of unlabeled competitor oligonucleotide, indicating a specificity of the binding of NF-κB. (c) NF-κB binding complex determined in the cells transfected with an increasing amounts (1-4 μg) of SphK^{G82D} or TB2-SphK followed by TNF stimulation. (d) Stable transfected 293 cells overexpressing SphK, SphK^{G82D}, or empty vector were cotransfected with TRAF2 or pRK5 vector together with Ig-κB-luc reporter plasmid and Renilla luciferase control vector. 24 h after transfection, cells were stimulated with TNF (1 ng/ml) for 4 h and then the reporter gene activity was determined and normalized relative to Renilla luciferase activity. Data shown are mean of relative luciferase activity of one representative experiment done in quadruplicate. Similar results were obtained in four independent experiments.

FIG. 6 is an image of the effect of SphK on JNK activation. 293T cells were cotransfected the indicated amounts of expression vectors. After 48 h cells were stimulated with or without TNF (1 ng/ml) for 30 min. JNK activity was assayed as described under ‘Methods’. An HA immunoblot is shown in the bottom panel indicating equivalent levels of HA-JNK.

FIG. 7 is a graphical representation of the effect of SphK on TRAF2-mediated antiapoptosis. 293T cells were cotransfected with SphK, SphK^{G82D}, or an empty vector together with TRAF2 or ΔTRAF2 as indicated. 48 h after the transfection, cells were stimulated with TNF (10 ng/ml) in the absence (left panel) or presence (right panel) of cycloheximide (CHX, 1 μg/ml) for a further 18 h. Cell viability was then assessed by an MTT assay and expressed as a proportion of cells maintained in normal culture medium containing 10% of FCS. Data shown are mean (±S.A.) of one representative experiment done in triplicate.

FIG. 8 is a schematic representation of a model showing the interaction of TRAF2 with SphK bifurcating TNF signaling pathways. TRAF2 interacted with and subsequently increased SphK activity that specifically transduces TNF signaling to activation of NF-κB and antiapoptosis but not JNK activation.

FIG. 9 is a schematic representation of TRAF2-binding motifs (bold characters) in the amino acid sequence of human SphK (top panel). The left panel shows the consensus of known TRAF2-binding motifs based on the published structural information (Ye, H, et al., Immunity 7:715, 1997). The bottom panel is a schematic representation of the mutants of SphK that mutated the TRAF2-binding motifs (TB1-SphK and TB2-SphK).

### DETAILED DESCRIPTION OF THE INVENTION

The present invention is predicated, in part, on both the elucidation of the role of sphingosine kinase in TRAF mediated cytokine signalling pathways and the identification of a sphingosine kinase binding motif via which its interaction with TRAF2 occurs. These determinations now permit the rational design of therapeutic and/or prophylactic methods for treating conditions, such as those characterised by aberrant or unwanted TNF signalling. Further, there is facilitated the identification and/or design of agents which specifically interact with this binding motif thereby introducing a degree of specificity into the claimed modulatory methodology which achieves a reduction in side effects due to its capacity to selectively modulate TRAF mediated NF-κB activation as opposed to TRAF mediated JNK activation.

Accordingly, one aspect of the present invention provides a method of modulating cytokine-induced cellular activity, said method comprising contacting said cell with an effective amount of an agent for a time and under conditions sufficient to modulate the interaction of sphingosine kinase with a TRAF wherein inducing or otherwise agonising said association up-regulates said cellular activity and inhibiting or otherwise antagonising said association down-regulates said cellular activity.

Reference to “cytokine induced cellular activity” should be understood as a reference to any one or more of the functional activities which a cell is capable of performing as a result of cytokine stimulation.

Cytokines are protein hormones and reference to “cytokine” herein should be understood as a reference to any protein hormone or derivative, homologue, analogue, chemical equivalent or mimetic thereof including, but not limited to the interleukins, the colony stimulating factors, the interferons or INF. Preferably said cytokine is TNF.

The present invention more particularly provides a method of modulating TNF-induced cellular activity, said method comprising contacting said cell with an effective amount of an agent for a time and under conditions sufficient to modulate the interaction of sphingosine kinase with a TRAF wherein inducing or otherwise agonising said association up-regulates said cellular activity and inhibiting or otherwise antagonising said association down-regulates said cellular activity.

By “TNF” is meant all forms of TNF, including for example TNF-α and TNF-β and derivatives, homologues, analogues, chemical equivalents and mimetics thereof. Reference to “TNF” should also be understood to include reference to any isoforms which arise from alternative splicing of TNF mRNA or mutants or polymorphic variants of TNF. It should also be understood to include reference to any other molecule which exhibits TNF functional activity to the extent that the subject molecule mimics one or more TNF signalling events by inducing signalling through a TNF or TNF-like receptor.

Since the method of the present invention is directed to modulating a cellular activity by modulating an intracellular signalling event which has been induced as a result of the interaction of TNF with its receptor, this methodology can be applied to modulating such a cellular activity, irrespective of whether it has been induced by the interaction of TNF with a TNF receptor or the interaction of a TNF mimetic, such as a naturally occurring or non-naturally occurring mimetic or analogue, with the subject receptor. It is conceivable, for example, that there may be naturally or non-naturally occurring TNF mimetics (for example, toxins or drugs) which, if they were introduced into an individual, would induce unwanted TNF-like cellular activities due to their interaction with the TNF receptor. Accordingly, the present invention should be understood to extend to the modulation of such cellular activities which are herein defined as falling within the scope of being “TNF-induced”.

Reference to “TRAF” should be understood as a reference to the TRAF class of adaptor proteins. There are currently six known members of this class, termed TRAF1-6. Reference to “sphingosine kinase” should be understood as a reference to the molecule which is, inter alia, involved in the generation of sphingosine-1-phosphate during activation of the sphingosine kinase signalling pathway.

Accordingly, reference to “TRAF” and “sphingosine kinase” should be understood as a reference to all forms of these proteins and to derivatives, homologues, analogues, chemical equivalents or mimetics thereof. This includes, for example, any isoforms which arise from alternative splicing of the subject TRAF or sphingosine kinase mRNA or mutants or polymorphic variants of these proteins. Preferably, said TRAF is TRAF2 or derivative, homologue, analogue, chemical equivalent or mimetic thereof.

TRAF2 is the prototypical member of TRAF family. It can interact directly or indirectly with a number of TNF receptors to mediate the signal transduction of these receptors. TRAF2 can also interact with numerous intracellular proteins such as I-TRAF/TANK, RIP, NIK and the caspase inhibitors cIAPs, and thereby transduces signals required for the activation of the transcription factor NF-κB, the stress-activated protein kinase (SAPK or JNK) and anti-apoptosis. Structural studies have revealed the complexity and flexibility of TRAF2 as a signal junction.

The present invention therefore more particularly provides a method of modulating TNF induced cellular activity said method comprising contacting said cell with an effective amount of an agent for a time and under conditions sufficient to modulate the interaction of sphingosine kinase with TRAF2 wherein inducing or otherwise agonising said association up-regulates said cellular activity and inhibiting or otherwise antagonising said association down-regulates said cellular activity.

Without limiting the present invention to anyone theory or mode of action, TRAF2 is one of the major adaptor proteins to mediate cell activation, survival and anti-apoptosis induced by the TNF receptor superfamily. The engagement of TNF receptors causes their interaction directly or indirectly with TRAF2 and subsequently recruits downstream signals leading to activation of JNK, NF-κB and anti-apoptotic pathways. The physical interaction of sphingosine kinase with TRAF2 has now been determined to result in the production of sphingosine-1-phosphate. Sphingosine-1-phosphate is a second messenger which regulates cell growth, survival and proliferation both in vivo and in vitro. It has also been determined that the interaction of TRAF2 with sphingosine kinase is required for TRAF2 mediated activation of NF-κB but not JNK. Accordingly, it is thought that interaction of TRAF2 with sphingosine kinase is responsible for one of the arms of TRAF2 mediated signalling, namely the activation of NF-κB, but not the other arm of TRAF2 signalling, being activation of JNK. Accordingly, to the extent that the present invention is directed to modulation of TRAF2 mediated signalling, there is provided a valuable level of specificity wherein agents which modulate sphingosine kinase/TRAF2 interaction will lead to modulation only of NF-κB mediated cellular activities, which are initially induced by TNF, and not those mediated by the JNK signalling pathway. This provides a valuable level of specificity which may potentially reduce side effects which would otherwise have been observed had both arms of the TRAF2 signalling pathway being modulated.

As detailed hereinbefore, TNF is a highly pleiotropic cytokine which elicits a wide spectrum of cellular responses including, but not limited to, cell proliferation, apoptosis, anti-apoptosis and inflammatory and immunoregulatory responses. Without limiting the present invention to any one theory or mode of action, it is thought that the activities mediated by TRAF signalling are predominantly those of cell activation, anti-apoptosis and proliferation. Still without limiting the present invention to any one theory or mode of action, the inventors have determined that TRAF2 does not contain intrinsic catalytic activity and that protein-protein interactions are essential for TRAF2 to mediate the activation of downstream signals. In particular, it has been determined that sphingosine kinase and TRAF2 interact via a sphingosine kinase binding motif in order to facilitate the induction of downstream signalling events, such as NK-κB activation. Reference to the “interaction” of sphingosine kinase and TRAF2 should therefore be understood as a reference to any form of interaction between these two molecules including, but not limited to, via hydrogen bonds, ionic bonds, van de Waals forces, covalent bonds or an other form of association.

Elucidation of both the role of sphingosine kinase in relation to TRAF mediated TNF stimulation and the nature of the interaction between these two molecules, now provides a mechanism for modulating TNF induced cellular activity. By “modulated” is meant up-regulated or down-regulated. For example, inducing or otherwise agonising the interaction of sphingosine kinase and TRAF provides a means of inducing or up-regulating the occurrence or degree of a TNF induced cellular activity which is mediated by the TRAF signalling pathway, for example up-regulation of proliferation and/or anti-apoptotic characteristics of a cell. Conversely, to the extent that a TNF induced cellular activity which is mediated by the TRAF signalling pathway is unwanted, for example unwanted cellular proliferation or cell survival, the method of the present invention now facilitates down-regulation of such activity via antagonism of the sphingosine kinase/TRAF interaction.

Reference to “inducing or otherwise agonising” should be understood as a reference to:
(i) inducing the interaction of sphingosine kinase with TRAF, in particular TRAF2, in order to effect TNF-like cellular activation; or
(ii) up-regulating, enhancing or otherwise agonising a sphingosine kinase/TRAF interaction subsequently to its initial induction.

Conversely, “inhibiting or otherwise antagonising” the interaction of sphingosine kinase with TRAF, in particular TRAF2, is a reference to:
(i) preventing the interaction of sphingosine kinase with TRAF (for example, subsequently to TNF stimulation or in a prophylactic capacity in the absence of any known or imminent TNF stimulation); or
(ii) antagonising an existing interaction of sphingosine kinase with TRAF such that it is ineffective or less effective (for example, reducing the binding affinity of these two molecules).

It should be understood that modulation of the interaction between sphingosine kinase and TRAF (either in the sense of up-regulation or down-regulation) may be partial or complete. Partial modulation occurs where only some of the sphingosine kinase/TRAF interactions which would normally occur in a given cell are affected by the method of the present invention (for example, the method of the present invention is applied to a cell for only part of the time that the cell is undergoing TNF stimulation or the agent which is contacted with the subject cell is provided in a concentration insufficient to saturate the intracellular sphingosine kinase/TRAF interactions) while complete modulation occurs where all sphingosine kinase/TRAF interactions are modulated.

Preferably, the subject TNF induced cellular activity is induction of anti-apoptotic characteristics or up-regulation of proinflammatory activity.

According to this preferred embodiment there is provided a method of down-regulating the TNF induced anti-apoptotic functional activity of a cell, said method comprising contacting said cell with an effective amount of an agent for a time and under conditions sufficient to down-regulate the interaction of sphingosine kinase with TRAF2 wherein inhibiting or otherwise antagonising said interaction up-regulates the apoptosis of said cell.

In another preferred embodiment there is provided a method of down-regulating the TNF-induced pro-inflammatory functional activity of a cell, said method comprising contacting said cell with an agent for a time and under conditions sufficient to down regulate the interaction of sphingosine kinase with TRAF2 wherein inhibiting or otherwise antagonising said interaction down-regulates one or more of said cells TNF induced pro-inflammatory activities.

Modulation of the interaction between sphingosine kinase and a TRAF may be achieved by any one of a number of techniques including, but not limited to:
(i) introducing into a cell a nucleic acid molecule encoding TRAF or sphingosine kinase or derivative, homologue or analogue thereof or introducing the proteinaceous form of sphingosine kinase or a TRAF or derivative, homologue, analogue, chemical equivalent or mimetic thereof in order to modulate the intracellular concentrations of TRAF or sphingosine kinase which are available for signalling purposes.
(ii) introducing into a cell a proteinaceous or non-proteinaceous molecule which modulates the transcriptional and/or translational regulation of a gene, wherein said gene may be a TRAF gene or the sphingosine kinase gene.
1(iii) introducing into a cell a proteinaceous or non-proteinaceous molecule which antagonises the interaction between a TRAF and sphingosine kinase, such as a competitive inhibiter.
(iv) introducing into a cell a proteinaceous or non-proteinaceous molecule which agonises the interaction between a TRAF and sphingosine kinase.

Reference to “agent” should be understood as a reference to any proteinaceous or non-proteinaceous molecule which modulates the interaction of sphingosine kinase with a TRAF and includes, for example, the molecules detailed in points (i)-(iv), above. The subject agent may be linked, bound or otherwise associated with any proteinaceous or non-proteinaceous molecule. For example, it may be associated with a molecule which permits its targeting to a localised region.

Said proteinaceous molecule may be derived from natural, recombinant or synthetic sources including fusion proteins or following, for example, natural product screening. Said non-proteinaceous molecule may be derived from natural sources, such as for example natural product screening or may be chemically synthesised. The present invention contemplates chemical analogues of said sphingosine kinase or TRAF capable of acting as agonists or antagonists of the sphingosine kinase/TRAF interaction. Chemical agonists may not necessarily be derived from said sphingosine kinase or TRAF but may share certain conformational similarities. Alternatively, chemical agonists may be specifically designed to mimic certain physiochemical properties of said sphingosine kinase or TRAF. Antagonists may be any compound capable of blocking, inhibiting or otherwise preventing said sphingosine kinase and TRAF from interacting. Antagonists include monoclonal antibodies specific for said sphingosine kinase or TRAF, or parts of said sphingosine kinase, and antisense nucleic acids which prevent transcription or translation of genes or mRNA in the subject cells. Modulation of expression may also be achieved utilising antigens, RNA, ribosomes, DNAzymes, RNA aptamers, antibodies or molecules suitable for use in co-suppression. Screening methods suitable for use in identifying such molecules are described in more detail hereinafter.

Said proteinaceous or non-proteinaceous molecule may act either directly or indirectly to modulate the interaction of sphingosine kinase with TRAF. Said molecule acts directly if it associates with sphingosine kinase or TRAF molecules. Said molecule acts indirectly if it associates with a molecule other than sphingosine kinase or TRAF, which other molecule either directly or indirectly modulates the interaction of sphingosine kinase with TRAF. Accordingly, the method of the present invention encompasses regulation of the sphingosine kinase/TRAF interaction via the induction of a cascade of regulatory steps.

“Derivatives” include fragments, parts, portions, mutants, variants and mimetics from natural, synthetic or recombinant sources including fusion proteins. Parts or fragments include, for example, active regions of sphingosine kinase or TRAF. Derivatives may be derived from insertion, deletion or substitution of amino acids. Amino acid insertional derivatives include amino and/or carboxylic terminal fusions as well as intrasequence insertions of single or multiple amino acids. Insertional amino acid sequence variants are those in which one or more amino acid residues are introduced into a predetermined site in the protein although random insertion is also possible with suitable screening of the resulting product. Deletional variants are characterized by the removal of one or more amino acids from the sequence. Substitutional amino acid variants are those in which at least one residue in the sequence has been removed and a different residue inserted in its place. An example of substitutional amino acid variants are conservative amino acid substitutions. Conservative amino acid substitutions typically include substitutions within the following groups: glycine and alanine; valine, isoleucine and leucine; aspartic acid and glutamic acid; asparagine and glutamine; serine and threonine; lysine and arginine; and phenylalanine and tyrosine. Additions to amino acid sequences include fusions with other peptides, polypeptides or proteins.

Reference to “homologues” should be understood as a reference to nucleic acid molecules or proteins derived from species other than the species being treated.

Chemical and functional equivalents of nucleic acid or protein molecules should be understood as molecules exhibiting any one or more of the functional activities of these molecules and may be derived from any source such as being chemically synthesized or identified via screening processes such as natural product screening.

The derivatives include fragments having particular epitopes or parts of the entire protein fused to peptides, polypeptides or other proteinaceous or non-proteinaceous molecules.

Analogues contemplated herein include, but are not limited to, modification to side chains, incorporating of unnatural amino acids and/or their derivatives during peptide, polypeptide or protein synthesis and the use of crosslinkers and other methods which impose conformational constraints on the proteinaceous molecules or their analogues.

Derivatives of nucleic acid sequences may similarly be derived from single or multiple nucleotide substitutions, deletions and/or additions including fusion with other nucleic acid molecules. The derivatives of the nucleic acid molecules of the present invention include oligonucleotides, PCR primers, antisense molecules, molecules suitable for use in cosuppression and fusion of nucleic acid molecules. Derivatives of nucleic acid sequences also include degenerate variants.

Examples of side chain modifications contemplated by the present invention include modifications of amino groups such as by reductive alkylation by reaction with an aldehyde followed by reduction with NaBH₄; amidination with methylacetimidate; acylation with acetic anhydride; carbamoylation of amino groups with cyanate; trinitrobenzylation of amino groups with 2, 4, 6-trinitrobenzene sulphonic acid (TNBS); acylation of amino groups with succinic anhydride and tetrahydrophthalic anhydride; and pyridoxylation of lysine with pyridoxal-5-phosphate followed by reduction with NaBH₄.

The guanidine group of arginine residues may be modified by the formation of heterocyclic condensation products with reagents such as 2,3-butanedione, phenylglyoxal and glyoxal.

The carboxyl group may be modified by carbodiimide activation via O-acylisourea formation followed by subsequent derivitisation, for example, to a corresponding amide.

Sulphydryl groups may be modified by methods such as carboxymethylation with iodoacetic acid or iodoacetamide; performic acid oxidation to cysteic acid; formation of a mixed disulphides with other thiol compounds; reaction with maleimide, maleic anhydride or other substituted maleimide; formation of mercurial derivatives using 4-chloromercuribenzoate, 4-chloromercuriphenylsulphonic acid, phenylmercury chloride, 2-chloromercuri-4-nitrophenol and other mercurials; carbamoylation with cyanate at alkaline pH.

Tryptophan residues may be modified by, for example, oxidation with N-bromosuccinimide or alkylation of the indole ring with 2-hydroxy-5-nitrobenzyl bromide or sulphenyl halides. Tyrosine residues on the other hand, may be altered by nitration with tetranitromethane to form a 3-nitrotyrosine derivative.

Modification of the imidazole ring of a histidine residue may be accomplished by alkylation with iodoacetic acid derivatives or N-carboethoxylation with diethylpyrocarbonate.

Examples of incorporating unnatural amino acids and derivatives during protein synthesis include, but are not limited to, use of norleucine, 4-amino butyric acid, 4-amino-3-hydroxy-5-phenylpentanoic acid, 6-aminohexanoic acid, t-butylglycine, norvaline, phenylglycine, ornithine, sarcosine, 4-amino-3-hydroxy-6-methylheptanoic acid, 2-thienyl alanine and/or D-isomers of amino acids. A list of unnatural amino acids contemplated herein is shown in Table 2.

TABLE 2

| Non-conventional | | Non-conventional | |
|---|---|---|---|
| amino acid | Code | amino acid | Code |
| α-aminobutyric acid | Abu | L-N-methylalanine | Nmala |
| α-amino-α-methylbutyrate | Mgabu | L-N-methylarginine | Nmarg |
| aminocyclopropane- | Cpro | L-N-methylasparagine | Nmasn |
| carboxylate | | L-N-methylaspartic acid | Nmasp |
| aminoisobutyric acid | Aib | L-N-methylcysteine | Nmcys |
| aminonorbornyl- | Norb | L-N-methylglutamine | Nmgln |
| carboxylate | | L-N-methylglutamic acid | Nmglu |
| cyclohexylalanine | Chexa | L-N-methylhistidine | Nmhis |
| cyclopentylalanine | Cpen | L-N-methylisolleucine | Nmile |
| D-alanine | Dal | L-N-methylleucine | Nmleu |
| D-arginine | Darg | L-N-methyllysine | Nmlys |
| D-aspartic acid | Dasp | L-N-methylmethionine | Nmmet |
| D-cysteine | Dcys | L-N-methylnorleucine | Nmnle |
| D-glutamine | Dgln | L-N-methylnorvaline | Nmnva |
| D-glutamic acid | Dglu | L-N-methylornithine | Nmorn |
| D-histidine | Dhis | L-N-methylphenylalanine | Nmphe |
| D-isoleucine | Dile | L-N-methylproline | Nmpro |
| D-leucine | Dleu | L-N-methylserine | Nmser |
| D-lysine | Dlys | L-N-methylthreonine | Nmthr |
| D-methionine | Dmet | L-N-methyltryptophan | Nmtrp |
| D-ornithine | Dorn | L-N-methyltyrosine | Nmtyr |
| D-phenylalanine | Dphe | L-N-methylvaline | Nmval |
| D-proline | Dpro | L-N-methylethylglycine | Nmetg |
| D-serine | Dser | L-N-methyl-t-butylglycine | Nmtbug |
| D-threonine | Dthr | L-norleucine | Nle |
| D-tryptophan | Dtrp | L-norvaline | Nva |
| D-tyrosine | Dtyr | α-methyl-aminoisobutyrate | Maib |
| D-valine | Dval | α-methyl--aminobutyrate | Mgabu |
| D-α-methylalanine | Dmala | α-methylcyclohexylalanine | Mchexa |
| D-α-methylarginine | Dmarg | α-methylcylcopentylalanine | Mcpen |
| D-α-methylasparagine | Dmasn | α-methyl-α-napthylalanine | Manap |
| D-α-methylaspartate | Dmasp | α-methylpenicillamine | Mpen |
| D-α-methylcysteine | Dmcys | N-(4-aminobutyl)glycine | Nglu |
| D-α-methylglutamine | Dmgln | N-(2-aminoethyl)glycine | Naeg |
| D-α-methylhistidine | Dmhis | N-(3-aminopropyl)glycine | Norn |
| D-α-methylisoleucine | Dmile | N-amino-α-methylbutyrate | Nmaabu |
| D-α-methylleucine | Dmleu | α-napthylalanine | Anap |
| D-α-methyllysine | Dmlys | N-benzylglycine | Nphe |
| D-α-methylmethionine | Dmmet | N-(2-carbamylethyl)glycine | Ngln |
| D-α-methylornithine | Dmorn | N-(carbamylmethyl)glycine | Nasn |
| D-α-methylphenylalanine | Dmphe | N-(2-carboxyethyl)glycine | Nglu |
| D-α-methylproline | Dmpro | N-(carboxymethyl)glycine | Nasp |
| D-α-methylserine | Dmser | N-cyclobutylglycine | Ncbut |
| D-α-methylthreonine | Dmthr | N-cycloheptylglycine | Nchep |
| D-α-methyltryptophan | Dmtrp | N-cyclohexylglycine | Nchex |
| D-α-methyltyrosine | Dmty | N-cyclodecylglycine | Ncdec |
| D-α-methylvaline | Dmval | N-cylcododecylglycine | Ncdod |
| D-N-methylalanine | Dnmala | N-cyclooctylglycine | Ncoct |
| D-N-methylarginine | Dnmarg | N-cyclopropylglycine | Ncpro |
| D-N-methylasparagine | Dnmasn | N-cycloundecylglycine | Ncund |
| D-N-methylaspartate | Dnmasp | N-(2,2-diphenylethyl)glycine | Nbhm |
| D-N-methylcysteine | Dnmcys | N-(3,3-diphenylpropyl)glycine | Nbhe |
| D-N-methylglutamine | Dnmgln | N-(3-guanidinopropyl)glycine | Narg |
| D-N-methylglutamate | Dnmglu | N-(1-hydroxyethyl)glycine | Nthr |
| D-N-methylhistidine | Dnmhis | N-(hydroxyethyl))glycine | Nser |
| D-N-methylisoleucine | Dnmile | N-(imidazolylethyl))glycine | Nhis |
| D-N-methylleucine | Dnmleu | N-(3-indolylyethyl)glycine | Nhtrp |
| D-N-methyllysine | Dnmlys | N-methyl-γ-aminobutyrate | Nmgabu |
| N-methylcyclohexylalanine | Nmchexa | D-N-methylmethionine | Dnmmet |
| D-N-methylornithine | Dnmorn | N-methylcyclopentylalanine | Nmcpen |
| N-methylglycine | Nala | D-N-methylphenylalanine | Dnmphe |
| N-methylaminoisobutyrate | Nmaib | D-N-methylproline | Dnmpro |
| N-(1-methylpropyl)glycine | Nile | D-N-methylserine | Dnmser |
| N-(2-methylpropyl)glycine | Nleu | D-N-methylthreonine | Dnmthr |
| D-N-methyltryptophan | Dnmtrp | N-(1-methylethyl)glycine | Nval |
| D-N-methyltyrosine | Dnmtyr | N-methyla-napthylalanine | Nmanap |
| D-N-methylvaline | Dnmval | N-methylpenicillamine | Nmpen |
| γ-aminobutyric acid | Gabu | N-(p-hydroxyphenyl)glycine | Nhtyr |
| L-t-butylglycine | Tbug | N-(thiomethyl)glycine | Ncys |
| L-ethylglycine | Etg | penicillamine | Pen |
| L-homophenylalanine | Hphe | L-α-methylalanine | Mala |
| L-α-methylarginine | Marg | L-α-methylasparagine | Masn |
| L-α-methylaspartate | Masp | L-α-methyl-t-butylglycine | Mtbug |
| L-α-methylcysteine | Mcys | L-methylethylglycine | Metg |
| L-α-methylglutamine | Mgln | L-α-methylglutamate | Mglu |
| L-α-methylhistidine | Mhis | L-α-methylhomophenylalanine | Mhphe |
| L-α-methylisoleucine | Mile | N-(2-methylthioethyl)glycine | Nmet |
| L-α-methylleucine | Mleu | L-α-methyllysine | Mlys |
| L-α-methylmethionine | Mmet | L-α-methylnorleucine | Mnle |
| L-α-methylnorvaline | Mnva | L-α-methylornithine | Morn |
| L-α-methylphenylalanine | Mphe | L-α-methylproline | Mpro |
| L-α-methylserine | Mser | L-α-methylthreonine | Mthr |
| L-α-methyltryptophan | Mtrp | L-α-methyltyrosine | Mtyr |
| L-α-methylvaline | Mval | L-N-methylhomophenylalanin | Nmhphe |
| N-(N-(2,2-diphenylethyl) | Nnbhm | N-(N-(3,3-diphenylpropyl) | Nnbhe |
| carbamylmethyl)glycine | | carbamylmethyl)glycine | |
| 1-carboxy-1-(2,2-diphenyl-Nmbc | | | |
| ethylamino)cyclopropane | | | |

Crosslinkers can be used, for example, to stabilise 3D conformations, using homo-bifunctional crosslinkers such as the bifunctional imido esters having (CH₂)ₙ spacer groups with n=1 to n=6, glutaraldehyde, N-hydroxysuccinimide esters and hetero-bifunctional reagents which usually contain an amino-reactive moiety such as N-hydroxysuccinimide and another group specific-reactive moiety.

It should be understood that the cell which is treated according to the method of the present invention may be located ex vivo or in vivo. By “ex vivo” is meant that the cell has been removed from the body of a subject wherein the modulation of its activity will be initiated in vitro. For example, the cell may be a non-neoplastic cell which is to undergo long term culture and is therefore stimulated to undergo ongoing TNF induced proliferation, which proliferation is up-regulated by agonists of the sphingosine kinase/TRAF2 interaction. In accordance with the preferred aspects of the present invention, the cell may be a neoplastic cell, such as a malignant cell, located in vivo (such as in the colon) and the down-regulation of its growth will be achieved by applying the method of the present invention in vivo to down-regulate the level of sphingosine kinase/TRAF interaction thereby inducing apoptosis.

Without limiting the invention to any one theory or mode of action, the inventors have identified the TRAF2 binding motif of sphingosine kinase. Specifically, it has now been determined that the C-terminal residues of sphingosine kinase play an important role in the interaction of sphingosine kinase with TRAF2. Even more particularly, it has been determined that the TRAF2 binding motif of sphingosine kinase corresponds to the site defined by amino acids PPEE at position 379-382 of <400>1.

In another aspect the present invention provides a method of modulating cytokine induced cellular activity said method comprising contacting said cell with an effective amount of an agent for a time and under conditions sufficient to modulate the interaction of sphingosine kinase with a TRAF, which agent binds, links or otherwise associates with the C-terminal region of sphingosine kinase, wherein inducing or otherwise agonising said interaction up-regulates said cellular activity and inhibiting or otherwise antagonising said interaction down-regulates said cellular activity.

More particularly, the present invention provides a method of modulating TNF induced cellular activity said method comprising contacting said cell with an effective amount of an agent for a time and under conditions sufficient to modulate the interaction of sphingosine kinase with TRAF2, which agent binds, links or otherwise associates with the C-terminal region of sphingosine kinase, wherein inducing or otherwise agonising said interaction up-regulates said cellular activity and inhibiting or otherwise antagonising said interaction down-regulates said cellular activity.

In a preferred embodiment, said C-terminal region is defined by the amino acid sequence PPEE and, more particularly, said PPEE motif corresponds to the motif located at position numbers 379-382 of <400>1.

According to this preferred embodiment, there is provided a method of modulating TNF induced cellular activity said method comprising contacting said cell with an effective amount of an agent for a time and under conditions sufficient to modulate the interaction of sphingosine kinase with TRAF2, which agent binds, links or otherwise associates with the amino acid sequence PPEE at the C-terminal region of sphingosine kinase, wherein inducing or otherwise agonising said interaction up-regulates said cellular activity and inhibiting or otherwise antagonising said interaction down-regulates said cellular activity.

Still more particularly, said sequence is located at position numbers 379-382 of <400>1.

A further aspect of the present invention relates to the use of the invention in relation to the treatment and/or prophylaxis of disease conditions. Without limiting the present invention to any one theory or mode of action, the pleiotropic activity of cytokines, and in particular TNF, renders these molecules an integral functional component of every aspect of both healthy and disease state physiological processes. Accordingly, the method of the present invention provides a valuable tool for modulating aberrant or otherwise unwanted cytokine functional activity.

Accordingly, another aspect of the present invention is directed to the method for the treatment and/or prophylaxis of a conditions characterised by aberrant, unwanted or otherwise inappropriate cytokine induced cellular activity in a mammal, said method comprising administering to said mammal an effective amount of an agent for a time and under conditions sufficient to modulate the interaction of sphingosine kinase with a TRAF wherein inducing or otherwise agonising said interaction up-regulates said cellular activity and inhibiting or otherwise antagonising said interaction down-regulates said cellular activity.

More particularly, there is provided a method for the treatment and/or prophylaxis of a condition characterised by aberrant, unwanted or otherwise inappropriate TNF induced cellular activity in a mammal, said method comprising administering to said mammal an effective amount of an agent for a time and under conditions sufficient to modulate the interaction of sphingosine kinase with a TRAF wherein inducing or otherwise agonising said interaction up-regulates said cellular activity and inhibiting or otherwise antagonising said interaction down-regulates said cellular activity.

Preferably, said TRAF is TRAF2.

Reference to “aberrant, unwanted or otherwise inappropriate” cellular activity should be understood as a reference to overactive cellular activity, to physiologically normal cellular activity which is inappropriate in that it is unwanted or to insufficient cellular activity. For example, TNF production during tumor cell growth has been shown to support cellular proliferation and to provide anti-apoptotic characteristics to the neoplastic cells. Without limiting the present invention in any way, it is thought that these pro-survival characteristics are signalled via the TRAF2 pathway. Clearly, to the extent that a cell is neoplastic, it is desirable that such activity be down-regulated. Similarly, diseases which are characterised by inflammation, such as rheumatoid arthritis, are known to involve TNF induced cellular activation, leading to the synthesis and secretion of inflammatory mediators. In such a situation, it is also desirable to down-regulate such activity. In yet another example, one of the current limitations associated with TNF therapy, for the treatment of neoplastic conditions, relates to the pleiotropic activities and multiple signalling pathways which are associated with TNF stimulation. Accordingly, although TNF stimulation may lead to activation of apoptotic pathways via the TRADD mediated signalling mechanism, a side effect of such treatment may be the TRAF mediated induction of inflammatory mediator production, which often leads to very serious inflammatory responses. Accordingly, the present invention now provides a means of tailoring TNF therapy in respect of neoplastic conditions. Specifically, whereas the administration of TNF can be utilised to induce neoplastic cell apoptosis, the simultaneous inhibition of TRAF2/sphingosine kinase interactions can be utilised to down-regulate the production of inflammatory mediators which may otherwise be induced. There is further achieved down-regulation of the anti-apoptotic characteristics which can be induced via TNF stimulation of the TRAF2 pathway. Accordingly, the method of the present invention provides a means of enhancing the killing activity of TNF and simultaneously decreasing unwanted inflammatory reactions.

Accordingly, in one preferred embodiment there is provided a method for the treatment and/or prophylaxis of a neoplastic condition said method comprising administering to said mammal an effective amount of an agent for a time and under conditions sufficient to down-regulate the interaction of sphingosine kinase with TRAF2 wherein inhibiting or otherwise antagonising said interaction down-regulates TNF induced anti-apoptotic functional characteristics.

In another preferred embodiment there is provided a method for the treatment and/or prophylaxis of inflammation in a mammal, said method comprising administering to said mammal an effective amount of an agent for a time and under conditions sufficient to down-regulate the interaction of sphingosine kinase with TRAF2 wherein inhibiting or otherwise antagonising said interaction down-regulates the production of TNF induced inflammatory mediators by said mammal.

The term “mammal” as used herein includes humans, primates, livestock animals (eg. sheep, pigs, cattle, horses, donkeys), laboratory test animals (eg. mice, rabbits, rats, guinea pigs), companion animals (eg. dogs, cats) and captive wild animals (eg. foxes, kangaroos, deer). Preferably, the mammal is human or a laboratory test animal Even more preferably, the mammal is a human.

An “effective amount” means an amount necessary at least partly to attain the desired response, or to delay the onset or inhibit progression or halt altogether, the onset or progression of a particular condition being treated. The amount varies depending upon the health and physical condition of the individual to be treated, the taxonomic group of individual to be treated, the degree of protection desired, the formulation of the composition, the assessment of the medical situation, and other relevant factors. It is expected that the amount will fall in a relatively broad range that can be determined through routine trials.

Reference herein to “treatment” and “prophylaxis” is to be considered in its broadest context. The term “treatment” does not necessarily imply that a subject is treated until total recovery. Similarly, “prophylaxis” does not necessarily mean that the subject will not eventually contract a disease condition. Accordingly, treatment and prophylaxis include amelioration of the symptoms of a particular condition or preventing or otherwise reducing the risk of developing a particular condition. The term “prophylaxis” may be considered as reducing the severity or onset of a particular condition. “Treatment” may also reduce the severity of an existing condition.

The present invention further contemplates a combination of therapies, such as the administration of the agent together with subjection of the mammal to circulating cytotoxic agents or to radiotherapy in the treatment of cancer.

Administration of the modulatory agent, in the form of a pharmaceutical composition, may be performed by any convenient means. The modulatory agent of the pharmaceutical composition is contemplated to exhibit therapeutic activity when administered in an amount which depends on the particular case. The variation depends, for example, on the human or animal and the modulatory agent chosen. A broad range of doses may be applicable. Considering a patient, for example, from about 0.1 mg to about 1 mg of modulatory agent may be administered per kilogram of body weight per day.

Dosage regimes may be adjusted to provide the optimum therapeutic response. For example, several divided doses may be administered daily, weekly, monthly or other suitable time intervals or the dose may be proportionally reduced as indicated by the exigencies of the situation.

The modulatory agent may be administered in a convenient manner such as by the oral, intravenous (where water soluble), intraperitoneal, intramuscular, subcutaneous, intradermal or suppository routes or implanting (e.g. using slow release molecules). The modulatory agent may be administered in the form of pharmaceutically acceptable nontoxic salts, such as acid addition salts or metal complexes, e.g. with zinc, iron or the like (which are considered as salts for purposes of this application). Illustrative of such acid addition salts are hydrochloride, hydrobromide, sulphate, phosphate, maleate, acetate, citrate, benzoate, succinate, malate, ascorbate, tartrate and the like. If the active ingredient is to be administered in tablet form, the tablet may contain a binder such as tragacanth, corn starch or gelatin; a disintegrating agent, such as alginic acid; and a lubricant, such as magnesium stearate.

Routes of administration include, but are not limited to, respiratorally, intratracheally, nasopharyngeally, intravenously, intraperitoneally, subcutaneously, intracranially, intradermally, intramuscularly, intraoccularly, intrathecally, intracereberally, intranasally, infusion, orally, rectally, via IV drip patch and implant.

In accordance with these methods, the agent defined in accordance with the present invention may be coadministered with one or more other compounds or molecules. By “coadministered” is meant simultaneous administration in the same formulation or in two different formulations via the same or different routes or sequential administration by the same or different routes. For example, the subject agent may be administered together with an agonistic agent in order to enhance its effects. By “sequential” administration is meant a time difference of from seconds, minutes, hours or days between the administration of the two types of molecules. These molecules may be administered in any order.

Another aspect of the present invention contemplates the use of an agent, as hereinbefore defined, in the manufacture of medicament for the treatment of a condition in a mammal, which condition is characterised by aberrant, unwanted or otherwise inappropriate cytokine induced cellular activity, wherein said agent modulates the interaction of sphingosine kinase with a TRAF and wherein inducing or otherwise agonising said interaction up-regulates said cellular activity and inhibiting or otherwise antagonising said interaction down-regulates said cellular activity.

Preferably, said cytokine is TNF and said TRAF is TRAF2.

Even more preferably, said modulation of TNF induced cellular activity is down-regulation of anti-apoptotic characteristics or down-regulation of the production of inflammatory mediators.

In yet another further aspect, the present invention contemplates a pharmaceutical composition comprising the modulatory agent as hereinbefore defined together with one or more pharmaceutically acceptable carriers and/or diluents. Said agents are referred to as the active ingredients.

The pharmaceutical forms suitable for injectable use include sterile aqueous solutions (where water soluble) or dispersions and sterile powders for the extemporaneous preparation of sterile injectable solutions or dispersion or may be in the form of a cream or other form suitable for topical application. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol and liquid polyethylene glycol, and the like), suitable mixtures thereof, and vegetable oils. The proper fluidity can be maintained, for example, by the use of a coating such as lecithin, by the maintenance of the required particle size in the case of dispersion and by the use of superfactants. The preventions of the action of microorganisms can be brought about by various antibacterial and antifungal agents, for example, parabens, chlorobutanol, phenol, sorbic acid, thimerosal and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various of the other ingredients enumerated above, as required, followed by filtered sterilisation. Generally, dispersions are prepared by incorporating the various sterilised active ingredient into a sterile vehicle which contains the basic dispersion medium and the required other ingredients from those enumerated above. In the case of sterile powders for the preparation of sterile injectable solutions, the preferred methods of preparation are vacuum drying and the freeze-drying technique which yield a powder of the active ingredient plus any additional desired ingredient from previously sterile-filtered solution thereof.

When the active ingredients are suitably protected they may be orally administered, for example, with an inert diluent or with an assimilable edible carrier, or it may be enclosed in hard or soft shell gelatin capsule, or it may be compressed into tablets, or it may be incorporated directly with the food of the diet. For oral therapeutic administration, the active compound may be incorporated with excipients and used in the form of ingestible tablets, buccal tablets, troches, capsules, elixirs, suspensions, syrups, wafers, and the like. Such compositions and preparations should contain at least 1% by weight of active compound. The percentage of the compositions and preparations may, of course, be varied and may conveniently be between about 5 to about 80% of the weight of the unit.

The amount of active compound in such therapeutically useful compositions in such that a suitable dosage will be obtained. Preferred compositions or preparations according to the present invention are prepared so that an oral dosage unit form contains between about 0.1 μg and 2000 mg of active compound.

The tablets, troches, pills, capsules and the like may also contain the components as listed hereafter: a binder such as gum, acacia, corn starch or gelatin; excipients such as dicalcium phosphate; a disintegrating agent such as corn starch, potato starch, alginic acid and the like; a lubricant such as magnesium stearate; and a sweetening agent such as sucrose, lactose or saccharin may be added or a flavouring agent such as peppermint, oil of wintergreen, or cherry flavouring. When the dosage unit form is a capsule, it may contain, in addition to materials of the above type, a liquid carrier. Various other materials may be present as coatings or to otherwise modify the physical form of the dosage unit. For instance, tablets, pills, or capsules may be coated with shellac, sugar or both. A syrup or elixir may contain the active compound, sucrose as a sweetening agent, methyl and propylparabens as preservatives, a dye and flavouring such as cherry or orange flavour. Of course, any material used in preparing any dosage unit form should be pharmaceutically pure and substantially non-toxic in the amounts employed. In addition, the active compound(s) may be incorporated into sustained-release preparations and formulations.

The pharmaceutical composition may also comprise genetic molecules such as a vector capable of transfecting target cells where the vector carries a nucleic acid molecule encoding a modulatory agent. The vector may, for example, be a viral vector.

Yet another aspect of the present invention relates to the agent as hereinbefore defined, when used in the method of the present invention.

Screening for the modulatory agents hereinbefore defined can be achieved by any one of several suitable methods including, but in no way limited to, contacting a cell comprising sphingosine kinase and TRAF with an agent and screening for the modulation of sphingosine kinase/TRAF functional activity or modulation of the activity or expression of a downstream sphingosine kinase or TRAF cellular target such as NF-κB. Detecting such modulation can be achieved utilising techniques such as Western blotting, electrophoretic mobility shift assays and/or the readout of reporters of sphingosine kinase or TRAF activity such as luciferases, CAT and the like.

It should be understood that the sphingosine kinase or TRAF protein may be naturally occurring in the cell which is the subject of testing or the genes encoding them may have been transfected into a host cell for the purpose of testing. Further, the naturally occurring or transfected gene may be constitutively expressed—thereby providing a model useful for, inter alia, screening for agents which down-regulate sphingosine kinase TRAF interactivity or the gene may require activation—thereby providing a model useful for, inter alia, screening for agents which modulate sphingosine kinase TRAF interactivity under certain stimulatory conditions, such as phage-display and yeast two- or multi-hybrid screening. Further, to the extent that a sphingosine kinase nucleic acid molecule is transfected into a cell, that molecule may comprise the entire sphingosine kinase gene or it may merely comprise a portion of the gene such as the TRAF2 binding portion.

In another example, the subject of detection could be a downstream sphingosine kinase regulatory target, rather than sphingosine kinase itself, such as TRAF2 or NF-κB. Yet another example includes sphingosine kinase binding sites ligated to a minimal reporter. For example, modulation of sphingosine kinase TRAF interactivity can be detected by screening for the modulation of the downstream signalling components of a TNF stimulated cell. This is an example of a system where modulation of the molecules which sphingosine kinase and TRAF regulate the activity of, are monitored. Where the cell which is the subject of the screening system is a neoplastic cell, for example, modulation of sphingosine kinase TRAF interactivity could be detected by screening for the induction of apoptosis of that cell.

Accordingly, another aspect of the present invention provides a method for detecting an agent capable of modulating the interaction of TRAF with sphingosine kinase or its functional equivalent or derivative thereof said method comprising contacting a cell or extract thereof containing said sphingosine kinase and TRAF or its functional equivalent or derivative with a putative agent and detecting an altered expression phenotype associated with said interaction.

Reference to “sphingosine kinase” and “TRAF” should be understood as a reference to either the sphingosine kinase or TRAF expression product or to a portion or fragment of the sphingosine kinase or TRAF molecule, such as the TRAF2 binding region of the sphingosine kinase protein. In this regard, the sphingosine kinase or TRAF expression product is expressed in a cell. The cell may be a host cell which has been transfected with the sphingosine kinase or TRAF nucleic acid molecule or it may be a cell which naturally contains the sphingosine kinase gene. Reference to “extract thereof” should be understood as a reference to a cell free transcription system.

Reference to detecting an “altered expression phenotype associated with said interaction” should be understood as the detection of cellular changes associated with modulation of the interaction of sphingosine kinase with TRAF. These may be detectable, for example, as intracellular changes or changes observable extracellularly. For example, this includes, but is not limited to, detecting changes in downstream product levels or activities (e.g. NF-κB).

In a preferred embodiment, the present invention provides a method for detecting an agent capable of modulating the interaction of TRAF with sphingosine kinase or its functional equivalent or derivative thereof said method comprising contacting a cell containing said sphingosine kinase and TRAF or its functional equivalent or derivative with a putative agent and detecting an altered apoptosis profile associated with said interaction.

In yet another aspect the present invention provides a method for detecting an agent capable of binding or otherwise associating with the TRAF2 binding site of sphingosine kinase or functional equivalent or derivative thereof said method comprising contacting a cell containing said binding site or functional equivalent or derivative thereof with a putative agent and detecting an altered expression phenotype associated with modulation of the function of sphingosine kinase or its functional equivalent or derivative.

Reference to “TRAF2 binding site of sphingosine kinase” should be understood as a reference to the sphingosine kinase binding motif which interacts with TRAF2, as hereinbefore defined.

In addition to screening for agents which modulate the interaction of TRAF and sphingosine kinase utilising function based assays of the type described above, the identification of the TRAF2 binding motif of sphingosine kinase also facilitates the screening, analysis, rational design and/or modification of agents for modulating the interaction of TRAF and sphingosine kinase based on analysis of the physical interaction of a putative agent or lead compound with the subject binding site.

Specifically, knowledge of the nature and location of the binding site now facilitates analysis of the tertiary structure of sphingosine kinase, in terms of the structure of the binding site, by techniques such as X-ray crystallography.

Accordingly, another aspect of the present invention is directed to a method for analysing, designing and/or modifying an agent capable of interacting with the TRAF binding site of sphingosine kinase or derivative thereof and modulating at least one functional activity associated with said sphingosine kinase said method comprising contacting said sphingosine kinase or derivative thereof with a putative agent and assessing the degree of interactive complementarity of said agent with said binding site.

The TRAF binding site of sphingosine kinase is herein referred to as the “sphingosine kinase binding site”.

Preferably said sphingosine kinase binding site is defined by the amino acid sequence PPEE at position numbers 379-382 of <400>1.

It should be understood that the sphingosine kinase which is contacted with the putative agent for evaluation of interactive complementarity may be recombinantly produced.

However, it should also be understood that the subject sphingosine kinase may take the form of an image based on the binding site structure which has been elucidated, such as an electron density map, molecular models (including, but not limited to, stick, ball and stick, space filling or surface representation models) or other digital or non-digital surface representation models or image, which facilitates the analysis of sphingosine kinase site: agent interactions utilising techniques and software which would be known to those of skill in the art. For example, interaction analyses can be performed utilising techniques such as Biacore real-time analysis of on and off-rates and dissociation constants for binding of ligands (Gardsvoll et al, 1999; Hoyer-Hansen et al, 1997; Ploug, 1998; Ploug et al, 1994; 1995; 1998) and NMR perturbation studies Stephens et al, 1992).

Reference to “assessing the degree of interactive complementarity” of an agent with the subject sphingosine kinase binding site should be understood as a reference to elucidating any feature of interest including, but not limited to, the nature and/or degree of interaction between the subject sphingosine kinase binding site and an agent of interest. As detailed above, any suitable technique can be utilised. Such techniques would be known to the person of skill in the art and can be utilised in this regard. In terms of the nature of the subject interaction, it may be desirable to assess the types of interactive mechanisms which occur between specific residues of any given agent and those of the sphingosine kinase binding site (for example, peptide bonding or formation of hydrogen bonds, ionic bonds, van der Waals forces, etc.) and/or their relative strengths. It may also be desirable to assess the degree of interaction which occurs between an agent of interest and the subject sphingosine kinase binding site. For example, by analysing the location of actual sites of interaction between the subject agent and sphingosine kinase binding site it is possible to determine the quality of fit of the agent into this region of the sphingosine kinase binding site and the relative strength and stability of that binding interaction. For example, if it is the object that sphingosine kinase binding site functioning be blocked, an agent which interacts with the sphingosine kinase binding site such that it blocks or otherwise hinders (for example, sterically hinders or chemically or electrostatically repels) TRAF2 interaction will be sought. The form of association which is required in relation to modulating TRAF mediated sphingosine kinase functioning may not involve the formation of any chemical interactive bonding mechanism, as this is traditionally understood, but may involve a non-bonding mechanism such as the proximal location of a region of the agent relative to the subject binding region of the sphingosine kinase binding site, for example, to effect steric hindrance with respect to the binding of an activating molecule. Where the interaction takes the form of hindrance or the creation of other repulsive forces, this should nevertheless be understood as a form of “interaction” despite the lack of formation of any of the traditional forms of bonding mechanisms.

It should also be understood that the sphingosine kinase binding site which is utilised either in a physical form or as an image, as hereinbefore discussed, to assess the interactive complementarity of a putative agent may be a naturally occurring form of the sphingosine kinase binding site or it may be a derivative, homologue, analogue, mutant, fragment or equivalent thereof. The derivative, homologue, analogue, mutant, fragment or equivalent thereof may take either a physical or non-physical (such as an image) form.

The determination of sphingosine kinase binding regions facilitates determination of the three dimensional structure of the sphingosine kinase binding site and the identification and/or rational modification and design of agents which can be used to modulate TRAF mediated sphingosine kinase functioning.

Without limiting the application of the present invention in any way, the method of the present invention facilitates the analysis, design and/or modification of agents capable of interacting with the sphingosine kinase binding site. In this regard, reference to “analysis, design and/or modification” of an agent should be understood in its broadest sense to include:
(i) Randomly screening (for example, utilising routine high-throughput screening technology) to identify agents which exhibit some modulatory capacity with respect to TRAF mediated sphingosine kinase functional activity and then analysing the precise nature and magnitude of the agent's modulatory capacity utilising the method of this aspect of the present invention. In this regard, existing crystals could be soaked with said agents or co-crystalisation could be performed. A combination of modelling and synthetic modification of the local compound together with mutagenesis of the sphingosine kinase binding site could then be performed for example. In screening for agents which may modulate activity, standard methods of phage display and also combinatorial chemistry may be utilised (Goodson et al., 1994; Terrett., 2000). Such interaction studies can also be furthered utilising techniques such as the Biacore analysis and NMR perturbation studies. Such agents are often commonly referred to as “lead” agents in terms of the random screening of proteinaceous or non-proteinaceous molecules for their capacity to function either agonistically or antagonistically. Further, for example, binding affinity and specificity could be enhanced by modifying lead agents to maximise interactions with the sphingosine kinase binding site. Such analyses would facilitate the selection of agents which are the most suitable for a given purpose. In this way, the selection step is based not only on in vitro data but also on a technical analysis of sites of agent: sphingosine kinase interaction in terms of their frequency, stability and suitability for a given purpose. For example, such analysis may reveal that what appears to be an acceptable in vitro activity in respect of a randomly identified agent is in fact induced by a highly unstable interaction due to the presence of proximally located agent: sphingosine kinase sites which exhibit significant repulsive forces thereby de-stabilising the overall interaction between the agent and the sphingosine kinase. This would then facilitate the selection of another prospective lead compound, exhibiting an equivalent degree of in vitro activity, but which agent does not, upon further analysis, involve the existence of such de-stabilising repulsive forces.
   Screening for the modulatory agents herein defined can be achieved by any one of several suitable methods, including in silico methods, which would be well known to those of skill in the art and which are, for example, routinely used to randomly screen proteinaceous and non-proteinaceous molecules for the purpose of identifying lead compounds.
   These methods provide a mechanism for performing high throughput screening of putative modulatory agents such as the proteinaceous or non-proteinaceous agents comprising synthetic, recombinant, chemical and natural libraries.
(ii) The candidate or lead agent (for example, the agent identified in accordance with the methodology described in relation to point (i)) could be modified in order to maximise desired interactions (for example, binding affinity to specificity) with the sphingosine kinase and to minimise undesirable interactions (such as repulsive or otherwise de-stabilising interactions).
   Methods of modification of a candidate or lead agent in accordance with the purpose as defined herein would be well known to those of skill in the art. For example, a molecular replacement program such as Amore (Navaza, 1994) may be utilised in this regard. The method of the present invention also facilitates the mutagenesis of known signal inducing agents in order to ablate or improve signalling activity.
(iii) In addition to analysing fit and/or structurally modifying existing molecules, the method of the present invention also facilitates the rational design and synthesis of an agent, such as an agonistic or antagonistic agent, based on theoretically modelling an agent exhibiting the desired sphingosine kinase binding site interactive structural features followed by the synthesis and testing of the subject agent.

It should be understood that any one or more of applications (i)-(iii) above, may be utilised in identifying a particular agent.

In a related aspect, the present invention should be understood to extend to the agents identified utilising any of the methods hereinbefore defined. In this regard, reference to an agent should be understood as a reference to any proteinaceous or non-proteinaceous molecule which modulates at least one TRAF mediated functional activity.

The present invention is further defined by the following non-limiting examples:

### EXAMPLE 1

### Identification of a Role for Sphingosine Kinase 1N Signal Transduction by TRAF2

### Materials and Methods

Cells, Plasmids, Mutagenesis, and Transfections.

HEK 293T were obtained from the American Type Culture Collection and maintained in DMEM (GIBCO BRL), supplemented with 10% fatal calf serum (FCS). Human umbilical vein cells (HUVEC) were isolated and maintained as described (Xia et al., J. Biol. Chem. 274:33143-33147, 1999). Human SphK1 (SphK) cDNA (Genbank accession no. AF200328) was FLAG epitope-tagged at the 3′ end and subcloned into pcDNA3 vector (Invitrogen) as described previously (Pitson et al., Biochem. J. 350 Pt. 2:429-441, 2000). For generation of SphK mutants, the FLAG-tagged SphK was cloned with pALTER (Promega) site directed mutagenic vector. Single stranded DNA was prepared and used as a template for oligonucleotide directed mutagenesis as detailed in the manufacturer's protocol. The mutagenic oligonucleotides (5′-TGCCACTGGCGGCGCCAGTGCC-3′ (<400>31) and 5′-CACCGCCAGCGGCGCCCTTAGA-3′ (<400>32)) were designed to generate the TB1-SphK and TB2-SphK mutants respectively and in combination for TB1/2-SphK. The mutants were sequenced to verify incorporation of the desired modifications and then subcloned into pcDNA3 vector. Generation of SphK^{G82D} was described previously (Pitson et al., J. Biol. Chem. 275:33945, 2000). Expression plasmids of pRK5-TRAF2-FLAG and pRK5-TRAF2₈₇₋₅₀₁-FLAG were gifts from Dr V. Dixit (Genentech Inc., South San Francisco). Lipofectamine 2000 (GIBCO BRL) was used for transient transfections according to the manufacturer's protocols.

Immunoprecipitations and Immunoblot Assays.

Transfected 293T cells from each 10 cm dish were lysed in 1 ml of lysis buffer (50 mM HEPES, 150 mM NaCl, 5 mM EDTA, 0.1% NP-40/Triton X-100, 20 mM NaF, 1 mM sodium orthovanadate, 10 μg/ml leupeptin and aprotinin). The lysates equalized a same amount of proteins were immunoprecipitated with anti-FLAG, anti-HA or control mouse IgG1 monoclonal antibodies (Sigma) for 2 hr at 4° C., respectively. The immune complexes were precipitated by incubation with Protein A/G PLUS-agarose beads (Santa Cruz) for another 2 hr. The agarose beads were washed twice with 1 ml of lysis buffer, twice with 1 ml of high salt (1M NaCl) lysis buffer, and twice more with lysis buffer.

The immunoprecipitates were separated by 10% SDS-PAGE and transferred to Hybond-P (Amersham Pharmacia Biotech). Subsequent immunoblotting analyses were performed as described elsewhere (Xia et al., 1999, supra)0. Antibodies against FLAG-epitope (M2, Kodak), HA-epitope (Sigma), TRAF2, and I-κBα (Santa Cruz) were used at a 1:5,000, 1:2,500 and a 1:1,000 dilution, respectively, for immunoblotting assays.

GST Fusion Protein Binding Assay.

The human SphK cDNA was subcloned in-frame into the GST fusion protein expression vector, pGEX-1 (Amersham Pharmacia Biotech). Expression and purification of the derived GST-SphK fusion proteins were performed as described (Smith et al., Gene 67:31-40, 1988). Cell lysates from each T75 flask of HUVEC or 293T cells overexpressed with TRAF2 or ΔTRAF2 were incubated with 20 μl of a 1:1 slurry of glutathione-Sepharose beads bound to the GST-SphK or GST alone fusion proteins for 2 hr at 4° C. After six times extensive washes with lysis buffer, the coprecipitating proteins, along with whole lysates, were analyzed by an immunoblot assay with anti-TRAF2 antibodies.

Cell Viability Assay.

The transfected 293T cells were seeded on a 48-well plate at a density of 2×10⁴ cell/well and stimulated with TNF (10 ng/ml) in the presence or absence of cycloheximide (1 μg/ml) for 18 h. Cell viability was assessed by an MTT dye reduction assay and expressed as a proportion of cells maintained in normal culture medium as previously described (Xia et al., 1999, supra).

Kinase Activity Assays.

SphK activity was measured by incubating the cytosolic fraction with 5 μM sphingosine dissolved in 0.1% Triton X-100 and [γ³²P]ATP (1 mM, 0.5 mCi/ml) for 15 min at 37° C. as described previously (Xia et al., 1999, supra). JNK activity was measured by the immune complex kinase assay in anti-HA immunoprecipitates form the cells coexpressed with HA-tagged JNK. The activity of immunoprecipitated complex was determined by incubation with GST-c-jun (1-79) fusion protein as substrate as described (Xia et al., Proc. Natl. Acad. Science. USA. 95:14196, 1998).

Electrophoretic Mobility Shift Assay.

293T cells were cotransfected the desired expression vectors or empty vector. Nuclear extracts were prepared 48 h after transfection followed by TNF stimulation. The double-stranded oligonucleotides used as a probe in these experiments include 5′-GGATGCCATTGGGGATTTCCTCTTTACTGGATGT-3′ (<400>33) which contains a consensus NF-κB binding site in E-selectin promoter that is underlined. Gel mobility shift of a consensus NF-κB oligonucleotide was performed by incubating a ³²P-labelled NF-κB probe with 4 μg of nuclear proteins as described (Xia, et al., 1998, supra). The specific DNA-protein complexes were completely abolished by addition of a 50-fold molar excess of unlabelled NF-κB oligonucleotides.

Reporter Assay.

Stable transfected 293 cells overexpressing SphK, SphK^{G82D}, or empty vector were cotransfected with pRK5-TRAF2 or pRK5 vector together with Ig-KB-luciferase reporter gene plasmid (pTK81-IgK, 200 ng per transfection) and Renilla luciferase control vector (pRL, 20 ng per transfection). Total amounts of transfected DNA were kept constant by supplementing empty vector as needed. Cell extracts were prepared 24 hr after transfection, and reporter gene activity was determined by the dual-luciferase assay system (Promega) and normalized relative to Renilla luciferase activity.

### EXAMPLE 2

### Identification of a Role for Sphingosine Kinase in Signal Transduction by TRAF2

### Results

TRAF2 Activated SphK and Mediated TNF-Induced SphK Activation.

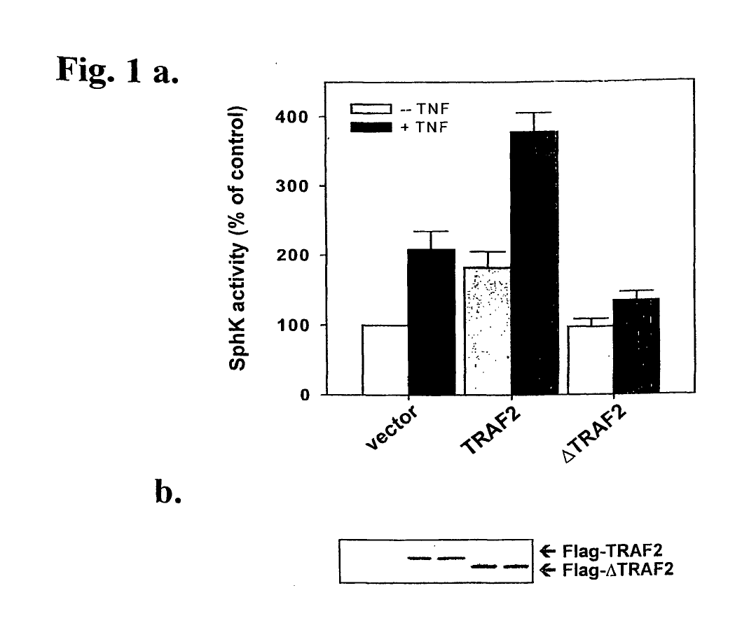

Testing is performed to determine whether TNF-induced SphK activation is mediated by TRAF2. Human embryonic kidney cell line 293T is transiently transfected with wild-type TRAF2, a dominant-negative TRAF2 (TRAF2₈₇₋₅₀₁, ΔTRAF2), or an empty vector. As shown in FIG. 1, overexpression of TRAF2 not only enhanced TNF-induced SphK activity, but also itself was capable of activating SphK by 2-fold compared with control transfectants. Immunoblotting assay showed equivalent expression levels of the transgenes in the presence or absence of TNF stimulation (FIG. 1b). In addition, the TNF-induced SphK activation was blocked by ΔTRAF2 containing a deletion of the N-terminal RING finger. These data indicate a role for TRAF2 in mediating TNF-induced SphK activation, a novel signaling pathway for cellular response to TNF stimulation.

TRAF2 Physically Interacted with SphK.

The possibility of a physical interaction between TRAF2 and SphK was examined. Initially, overexpression-based coimmunoprecipitation assays in HEK 293T cell line coexpressed HA-epitope-tagged SphK with FLAG-epitope-tagged TRAF2 or ΔTRAF2.

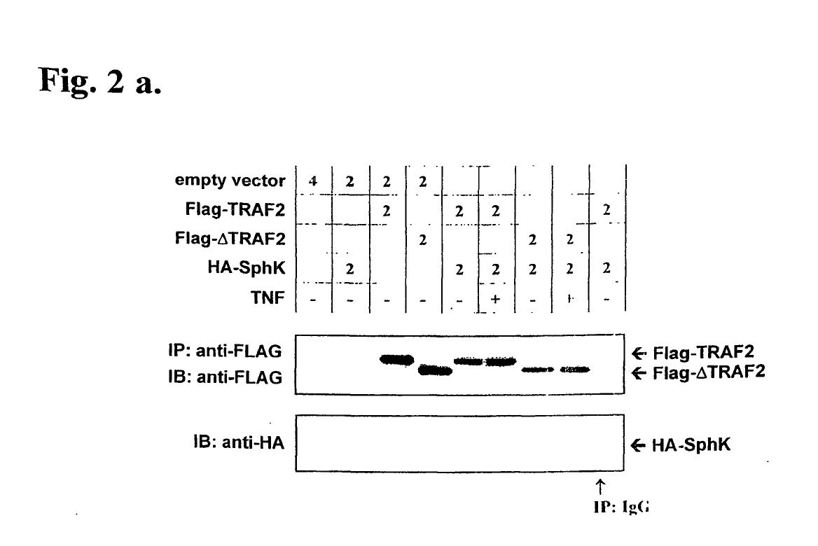

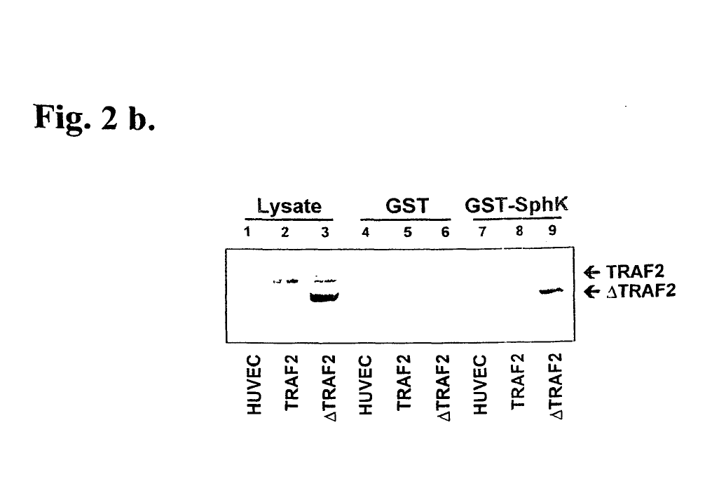

Were performed The cell lysates were immunoprecipitated with anti-FLAG monoclonal antibodies and the coprecipitated HA-tagged SphK was detected by immunoblot assay with anti-HA antibodies. SphK was found to be associated with TRAF2 in the immunoprecipitate complexes from the transfected cells (FIG. 2a). Conversely, by using anti-HA-epitope antibodies to perform the immunoprecipitation assays it was also found that FLAG-tagged TRAF2 or ΔTRAF2 was coprecipitated with HA-tagged SphK (data not shown). In addition, endogenous TRAF2 was examined in terms of whether it could also interact with SphK by using GST-SphK fusion protein to pull-down the associated cellular proteins. As shown in FIG. 2b, GST-SphK fusion protein was capable of interacting with not only the overexpressed TRAF2 in 293T cells, but also the endogenous TRAF2 in human endothelial cells (HUVEC), confirming a physical interaction of TRAF2 with SphK. The dominant-negative TRAF2 (ΔTRAF2) was also shown to be associated with SphK (FIG. 2), indicating that N-terminal RING finger of TRAF2 is not required for the interaction.

A TRAF2-Binding Motif, PPEE, is Responsible for the Interaction of TRAF2 with SphK.

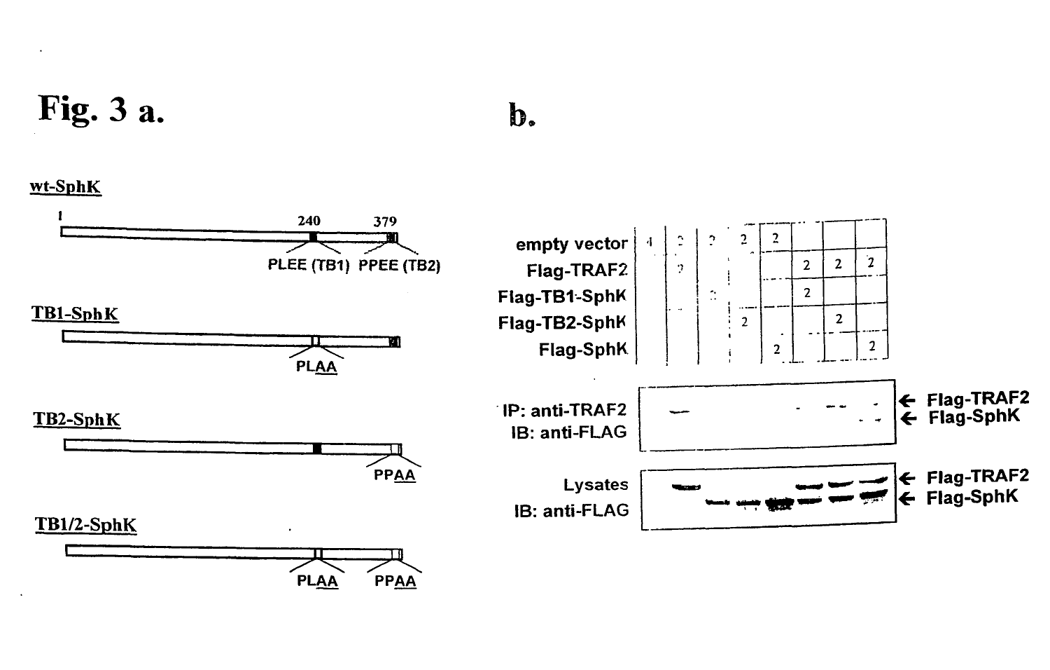

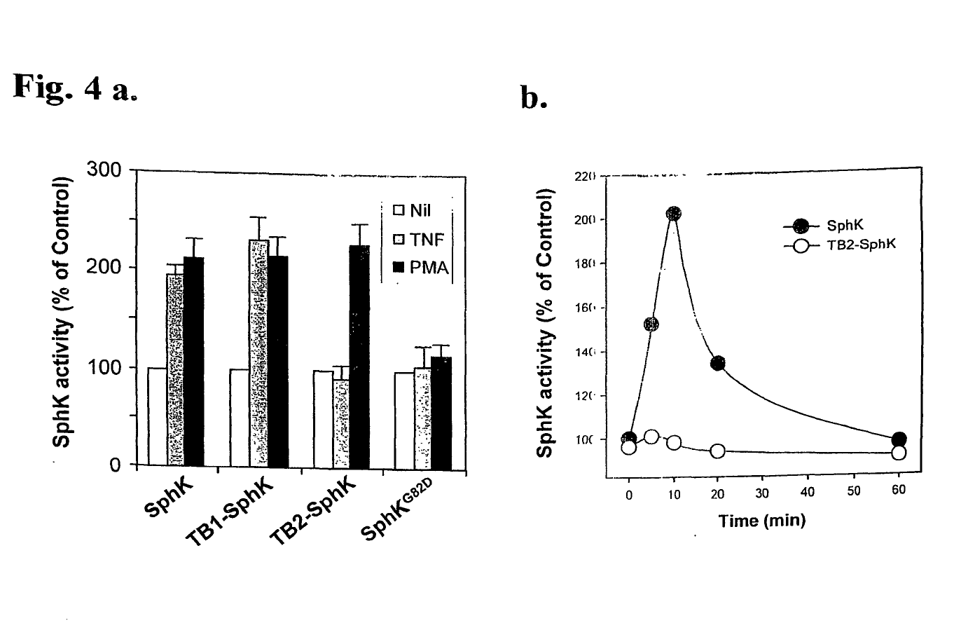

A structure based sequence alignment of TRAF2-binding sequences in various members of TNF receptor superfamily demonstrated a major consensus motif of (P/S/T/A)X(Q/E)E and minor motif of PXQXXD (Ye et al., Mol Cell 4:321-330, 1999; Ye et al., Proc. Natl. Acad. Sci. USA. 97:8961-8966, 2000). Analysis of the SphK sequence (human SphK-1) revealed two possible TRAF2-binding motifs in positions of 240-243 (PLEE) and 379-382 (PPEE), respectively, providing a potential structural basis for the interaction of SphK and TRAF2. To test if these two TRAF2-binding motifs are responsible for the binding of SphK to TRAF2, three mutants of SphK were generated: TB1-SphK, TB2-SphK and TB1/2-SphK in which the first, second or both TRAF2-binding motifs were mutated with alanines, ie, PLEE>PLAA and PPEE>PPAA, respectively (FIG. 3a). It was determined that expression of either TB2-SphK or TB1/2-SphK, but not TB1-SphK deleted the ability of SphK to coimmunoprecipitate with TRAF2 (FIG. 3b), indicating that the second TRAF2-binding motif is essential for the interaction of these two molecules. The cells enforced expressing TB1-SphK, TB2-SphK or TB1/2-SphK raised an unstimulated SphK activity to similar levels found with wild-type SphK-transfected cells (FIG. 4a), revealing an undiminished intrinsic enzyme catalytic activity in these SphK mutants. Strikingly, the activity of TB2-SphK, but not TB1-SphK, failed to respond to TNF stimulation (FIGS. 4a and 4b), suggesting an important role for C-terminal TB2 site of SphK not only in its capacity of interaction with TRAF2, but also in mediating TNF-induced activation of SphK. By contrast, the response of TB-2 SphK to phorbol ester (PMA), an activator of SphK through protein kinase C activation (Cuvillier et al., 1996, supra; Xia et al., 1999, supra), was undiminished (FIG. 4a), suggesting a TNF-specific defect of TB2-SphK. Taken together, these data indicate that SphK interacts with TRAF2 through the binding motif of PPEE₃₇₉₋₃₈₂, and that this interaction is responsible for mediating TNF-induced SphK activation.

Interaction of TRAF2 with SphK is Required for TNF-Induced NF-κB Activation.

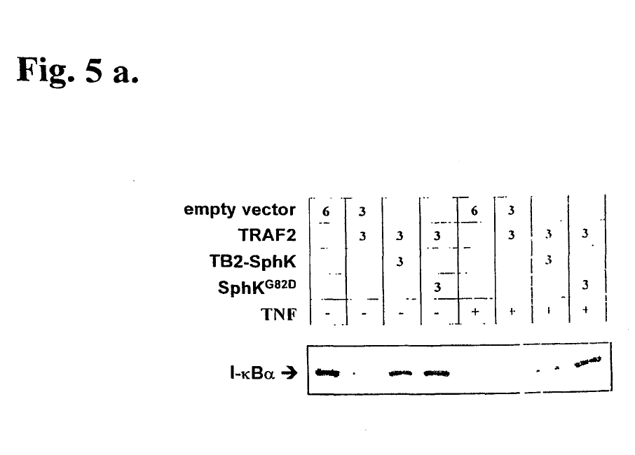

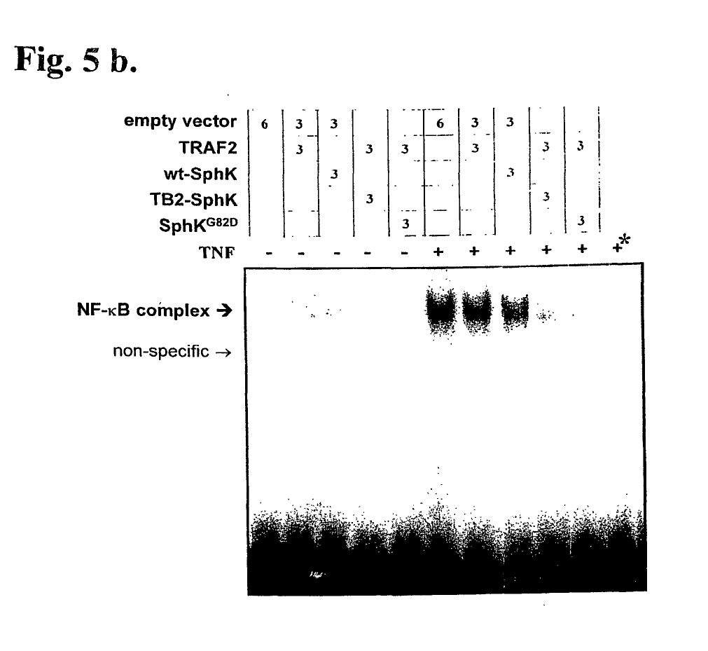

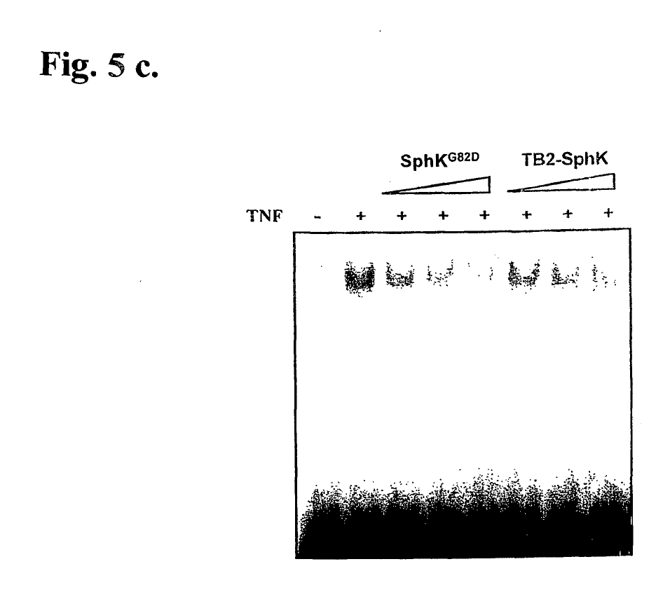

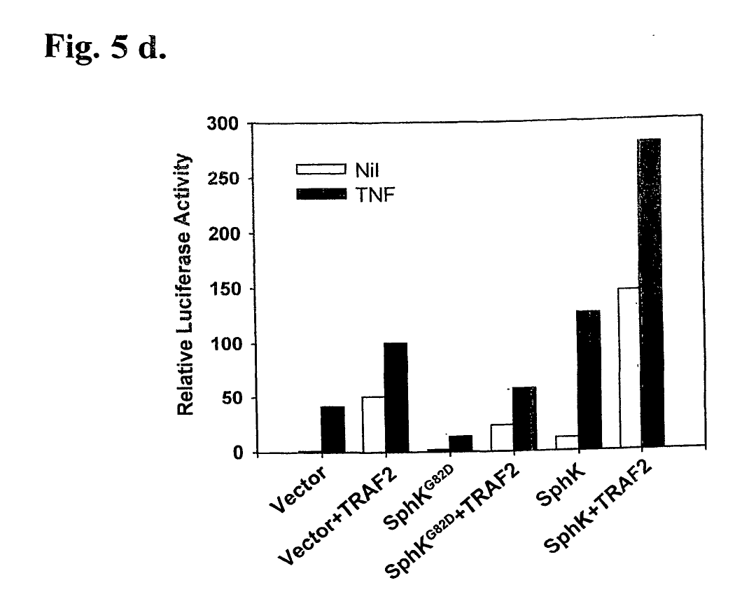

Given the fact that TRAF2 interacted with and subsequently activated SphK and that SphK has been implicated in signaling to regulate cell survival and activation (Cuvillier et al., 1996 supra; Xia et al., 2000, supra), it was sought to determine the role of SphK in the TRAF2-transduced signals. Overexpression of TRAF2 was capable of activating NF-κB as determined here by degradation of IκBα and gel shift assay of NF-κB DNA binding complex (FIGS. 4a and 4b). Coexpression of TB2-SphK markedly inhibited IκBα degradation (FIG. 5a) and decreased NF-κB DNA binding activity (FIGS. 5b and 5c) induced by either TNF stimulation or overexpression of TRAF2. By contrast, overexpression of wild-type SphK increased NF-κB activity (FIG. 5b), suggesting a potential effect of SphK on NF-κB activation.

To further establish the role of the interaction of SphK with TRAF2 in mediating TNF-induced NF-κB activation, a point mutant of SphK (SphK^{G82D}) that reserves intact the TRAF2-binding motif but lacks the enzyme catalytic activity (Pitson et al., 2000, supra) was used. SphK^{G82D} had undiminished binding ability to TRAF2 as determined by coimmunoprecipitation and completely abolished the SphK activity in response to TNF stimulation (FIG. 4a). Expression of SphK^{G82D} dramatically blocked the degradation of IκBα (FIG. 5a) and inhibited the NF-κB DNA binding activity in a dose-dependent manner (FIGS. 5b and 5c). NF-κB reporter gene assays confirmed the result from the assays of IκBα degradation and NF-κB DNA binding, showing that overexpression of TRAF2 or SphK increased NF-κB dependent gene activity, whereas the effect of TNF or TRAF2 on NF-κB activation was blocked by coexpression of SphK^{G82D} (FIG. 5d). Thus, the TRAF2-mediated SphK activation is apparently necessary for TNF-induced NF-κB activation.

Interaction of TRAF2 and SphK Does not Signal JNK Activation.

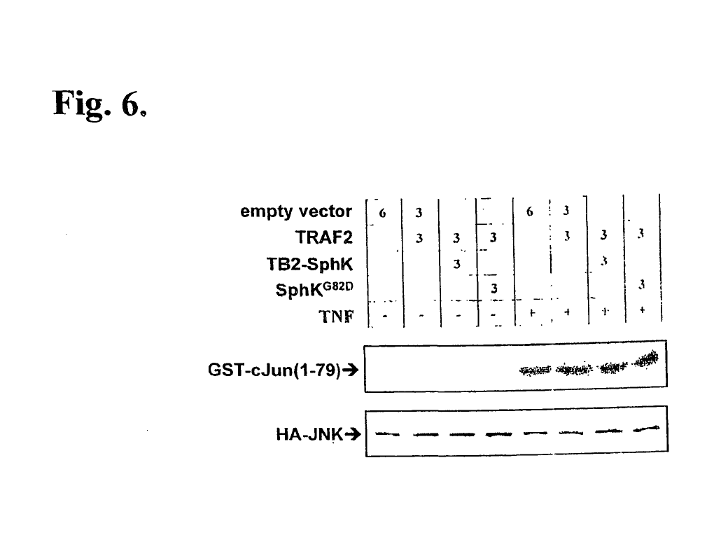

Since JNK is another well-documented major signal pathway mediated by TRAF2 during TNF stimulation (Lui et al., Cell 87:565-576, 1996; Lee et al., Immunity 7:703-713, 1997), the interaction of TRAF2 with SphK to regulate the TRAF2-dependent JNK activation was tested. Strikingly, neither TNF stimulation nor overexpression of TRAF2-induced JNK activity was affected by expression of TB2-SphK or SphK^{G82D} (FIG. 6). In addition, overexpression of wild-type SphK had no significant effect on JNK activation.

Hence, in contrast with the effect of SphK or NF-κB, the activation of JNK induced by TNF or TRAF2 is independent of SphK.

SphK Activation is Involved in TRAF2 Antiapoptotic Signaling.

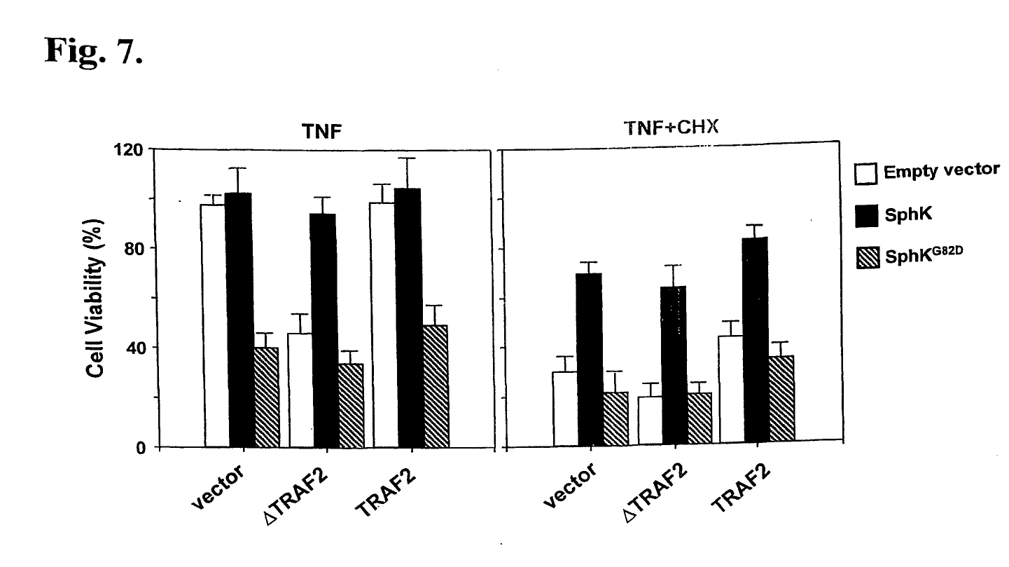

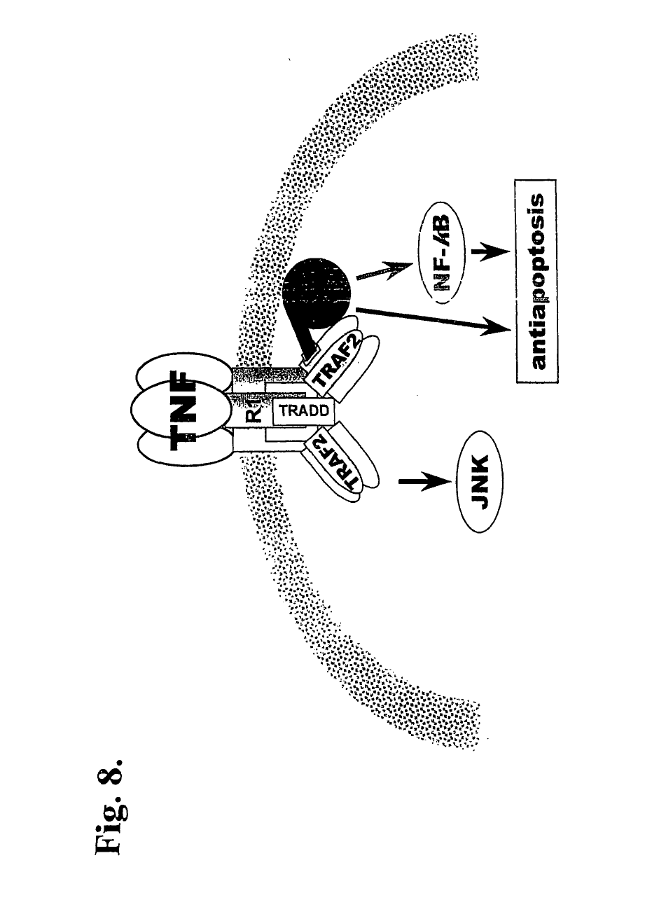

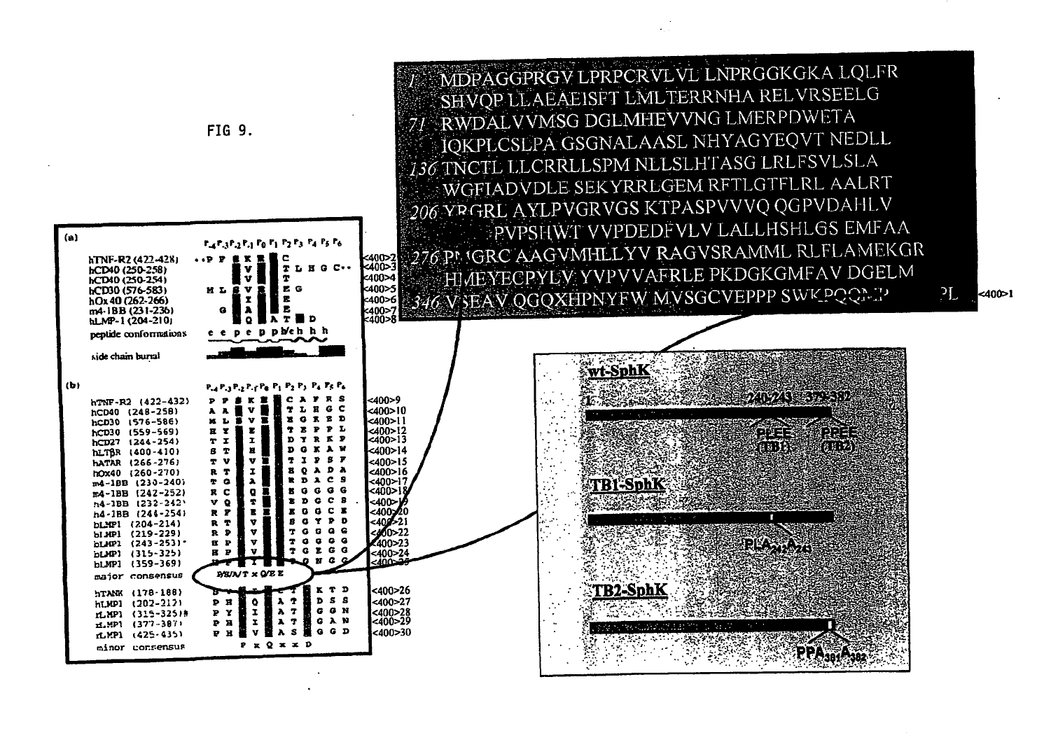

Investigations were performed as to whether the interaction of TRAF2 with SphK is involved in TRAF2-mediated antiapoptotic signaling pathways. Expression of ΔTRAF2 increased cell sensitivity to killing by TNF (FIG. 7), indicating the role of TRAF2 in antiapoptosis. The effect of ΔTRAF2 was completely prevented by overexpression of SphK even in the presence of an inhibitor of protein synthesis, cycloheximide (CHX), suggesting an independent of de novo protein synthesis antiapoptotic pathway promoted by SphK activation (FIG. 7). Whilst overexpression of TRAF2 had a partially protective effect against TNF-induced apoptosis in the presence of CHX, it was substantially enhanced by coexpression with SphK (FIG. 7, right panel). By contrast, the protective effect of TRAF2 against apoptosis was abolished by coexpression of SphK^{G82D} (FIG. 7). Taken together, these findings indicate that SphK activity is essential to determine the antiapoptotic capacity of TRAF2 during TNF stimulation.

Those skilled in the art will appreciate that the invention described herein is susceptible to variations and modifications other than those specifically described. It is to be understood that the invention includes all such variations and modifications. The invention also includes all of the steps, features, compositions and compounds referred to or indicated in this specification, individually7 or collectively, and any and all combinations of any two or more of said steps or features.

### BIBLIOGRAPHY

R. H. Arch, R. W. Gedrich, C. B. Thompson, Genes & Devel. 12, 2821 (1998)
A. M. Chinnaiyan et al., Science 274, 990 (1996)
O. Cuvillier et al., Nature 381, 800 (1996)
Gardsvoll et al, J Biol Chem 274(53), 37995-38003 (1999)
Goodson et al., Proc. Natl. Acad. Sci. USA 91(15), 7129-7133 (1994)
Y. A. Hannun, C. Luberto, K. M. Argraves, Biochemistry 40, 4893 (2001)
Y. A. Hannun, Science 274, 1855 (1996)
Hoyer-Hansen et al, FEBS Lett 420(1), 79-85 (1997)
H. Hsu, J. Xiong, D. V. Goeddel, Cell 81, 495 (1995)
S. Y. Lee et al. Immunity 7, 703 (1997)
Z. G. Liu, H. Hsu, D. V. Goeddel, M. Karin, Cell 87, 565 (1996)
R. M. Locksley, N. Killeen, M. J. Lenardo, Cell 104, 487 (2001)
Navaza, J. Acta Cryst A50, 157-163 (1994)
A. Olivera, S. Spiegel, Nature 365, 557 (1993)
Y. C. Park et al., Cell 101, 777 (2000)
S. M. Pitson et al., J. Biol. Chem. 275, 33945 (2000)
S. M. Pitson, R. J. D'andrea, L. Vandeleur, P. A. Moretti, P. Xia, J. R. Gamble, M. A. Vadas, and B. W. Wattenberg, Biochem. J. 350 Pt 2:429-441 (2000)
Ploug, M. Biochemistry 37(47), 16494-16505 (1998)
Ploug et al, Biochemistry 33(30), 8991-8997 (1994)
Ploug et al, Biochemistry 34(39), ₁₂₅2₄₋₁₂₅₃₄ (1995)
Ploug et al, Biochemistry 37(110), 3612-3522 (1998)
M. Rothe, M. G. Pan, W. J. Henzel, T. M. Ayres, D. V. Goeddel, Cell 83, 1243 (1995)
D. B. Smith and K. S. Johnson, Gene 67:31-40 (1998)
L. A. Tartaglia, M. Rothe, Y. F. Hu, D. V. Goeddel, Cell 73, 213 (1993)
Terrett, N. Drug Discov Today 5(5), 211-212 (2000)
C. Y. Wang, M. W. Mayo, R. G. Komeluk, D. V. Goeddel, A. S. Baldwin, Jr. Science 281, 1680 (1998)
P. Xia, L. Wang, J. R. Gamble, M. A. Vadas, J. Biol. Chem. 274, 34499 (1999)
P. Xia et al., Cur. Biol. 10, 1527 (2000)
P. Xia et al, Proc. Natl. Acad. Science. USA. 95, 14196 (1998)
P. Xia et al. J Biol Chem 274:33143-33147 (1999)
W.-C. Yeh et al. Immunity 7, 715 (1997)
H. Ye, Y. C. Park, M. Kreishman, E. Kieff and H. Wu, Mol Cell 4:321-330 (1999)
H. Ye and H. Wu, Proc Natl Acad Sci USA 97:8961-8966 (2000)

1. A method of modulating cytokine-induced cellular activity, said method comprising contacting said cell with an effective amount of an agent for a time and under conditions sufficient to modulate the interaction of sphingosine kinase with a TRAF wherein inducing or otherwise agonising said association up-regulates said cellular activity and inhibiting or otherwise antagonising said association down-regulates said cellular activity.

2. The method of claim 1, wherein said cytokine comprises Tumor Necrosis Factor (TNF).

3. The method of claim 1 wherein said TRAF is TRAF2.

4. The method according to claim 2 wherein said TNF-induced cellular activity is the induction of anti-apoptotic characteristics and said modulation is down-regulation of the interaction of sphingosine kinase with TRAF.

5. The method according to claim 2 wherein said TNF-induced cellular activity is the induction of pro-inflammatory activity and said induction is down-regulation of the interaction of sphingosine kinase with TRAF.

6. The method according to claim 1 wherein said agent binds, links or otherwise associates with the C-terminal region of sphingosine kinase.

7. The method according to claim 6 wherein said C-terminal region is defined by the amino acid sequence PPEE.

8. The method according to claim 7 wherein said sphingosine kinase is human sphingosine kinase and said C-terminal region is defined by the amino acid sequence PPEE at amino acid residue numbers 379-382 of <400>1.

9. A method for the treatment and/or prophylaxis of a condition characterised by aberrant, unwanted or otherwise inappropriate cytokine induced cellular activity in a mammal, said method comprising administering to said mammal an effective amount of an agent for a time and under conditions sufficient to modulate the interaction of sphingosine kinase with a TRAF wherein inducing or otherwise agonising said interaction up-regulates said cellular activity and inhibiting or otherwise antagonising said interaction down-regulates said cellular activity.

10. The method of claim 9, wherein said cytokine comprises TNF.

11. The method of claim 9, wherein said TRAF is TRAF2.

12. The method according to claim 10 wherein said TNF-induced cellular activity is the induction of anti-apoptotic characteristics and said modulation is down-regulation of the interaction of sphingosine kinase with TRAF.

13. The method according to claim 12 wherein said condition is a neoplastic condition.

14. The method according to claim 10 wherein said TNF-induced cellular activity is the induction of pro-inflammatory activity and said induction is down-regulation of the interaction of sphingosine kinase with TRAF.

15. The method according to claim 9 wherein said agent binds, links or otherwise associates with the C-terminal region of sphingosine kinase.

16. The method according to claim 15 wherein said C-terminal region is defined by the amino acid sequence PPEE.

17. The method according to claim 16 wherein said sphingosine kinase is human sphingosine kinase and said C-terminal region is defined by the amino acid sequence PPEE at amino acid residue numbers 379-382 of <400>1.

18. The method according to claim 9 wherein said mammal is a human.

19-23. (canceled)

24. The method according to claim 10 wherein said TNF-induced cellular activity is the induction of pro-inflammatory activity and said induction is down-regulation of the interaction of sphingosine kinase with TRAF.

25-28. (canceled)

29. A pharmaceutical composition comprising the modulatory agent defined in accordance with the methods of claim 1 together with one or more pharmaceutically acceptable carriers and/or diluents.

30. (canceled)

31. A method for detecting an agent capable of modulating the interaction of TRAF with sphingosine kinase or its functional equivalent or derivative thereof said method comprising contacting a cell or extract thereof containing said sphingosine kinase and TRAF or its functional equivalent or derivative with a putative agent and detecting an altered expression phenotype associated with said interaction.

32. The method according to claim 31 wherein said TRAF is TRAF2.

33. The method according to claim 32 wherein said altered expression phenotype is an altered apoptosis profile.

34. The method according to claim 31 wherein said altered expression phenotype is modulation of the functional activity of sphingosine kinase.

35. A method for analysing, designing and/or modifying an agent capable of interacting with the TRAF binding site of sphingosine kinase or derivative thereof and modulating at least one functional activity associated with said sphingosine kinase said method comprising contacting said sphingosine kinase or derivative thereof with a putative agent and assessing the degree of interactive complementarity of said agent with said binding site.

36. The method according to claim 35 wherein said TRAF binding site is the C-terminal region of sphingosine kinase.

37. The method according to claim 36 wherein said C-terminal region is defined by the amino acid sequence PPEE.

38. The method according to claim 37 wherein said sphingosine kinase is human sphingosine kinase and said C-terminal region is defined by the amino acid sequence PPEE at amino acid residue numbers 379-382 of <400>1.

39. The agent identified in accordance with claim 35.

40. The agent of claim 39 when used in the method of claim 1.


SEQUENCE LISTING (33 sequences)

SEQ ID NO: 1 - protein, 384 aa, Homo sapiens
  VARIANT 354: Xaa = Any Amino Acid
MDPAGGPRGVLPRPCRVLVLLNPRGGKGKALQLFRSHVQPLLAEAEISFTLMLTERRNHARELVRSEELGRWDALVVMSGDGLMHEVVNGLMERPDWETAIQKPLCSLPAGSGNALAASLNHYAGYEQVTNEDLLTNCTLLLCRRLLSPMNLLSLHTASGLRLFSVLSLAWGFIADVDLESEKYRRLGEMRFTLGTFLRLAALRTYRGRLAYLPVGRVGSKTPASPVVVQQGPVDAHLVPLEEPVPSHWTVVPDEDFVLVLALLHSHLGSEMFAAPMGRCAAGVMHLLYVRAGVSRAMMLRLFLAMEKGRHMEYECPYLVYVPVVAFRLEPKDGKGMFAVDGELMVSEAVQGQXHPNYFWMVSGCVEPPPSWKPQQMPPPEEPL

SEQ ID NO: 2 - protein, 7 aa, Unknown
  Consensus of known TRAF-2 binding motifs
PFSKEEC

SEQ ID NO: 3 - protein, 9 aa, Unknown
  Consensus of known TRAF-2 binding motifs
PVQETLHGC

SEQ ID NO: 4 - protein, 5 aa, Unknown
  Consensus of known TRAF-2 binding motifs
PVQET

SEQ ID NO: 5 - protein, 8 aa, Unknown
  Consensus of known TRAF-2 binding motifs
MLSVEEEG

SEQ ID NO: 6 - protein, 5 aa, Unknown
  Consensus of known TRAF-2 binding motifs
PIQEE

SEQ ID NO: 7 - protein, 6 aa, Unknown
  Consensus of known TRAF-2 binding motifs
GAAQEE

SEQ ID NO: 8 - protein, 7 aa, Unknown
  Consensus of known TRAF-2 binding motifs
PQQATDD

SEQ ID NO: 9 - protein, 11 aa, Unknown
  Consensus of known TRAF-2 binding motifs
PFSKEECAFRS

SEQ ID NO: 10 - protein, 11 aa, Unknown
  Consensus of known TRAF-2 binding motifs
AAPVQETLHGC

SEQ ID NO: 11 - protein, 11 aa, Unknown
  Consensus of known TRAF-2 binding motifs
MLSVEEEGKED

SEQ ID NO: 12 - protein, 11 aa, Unknown
  Consensus of known TRAF-2 binding motifs
HYPEQETEPPL

SEQ ID NO: 13 - protein, 11 aa, Unknown
  Consensus of known TRAF-2 binding motifs
TIPIQEDYRKP

SEQ ID NO: 14 - protein, 11 aa, Unknown
  Consensus of known TRAF-2 binding motifs
STPHQEDGKAW

SEQ ID NO: 15 - protein, 11 aa, Unknown
  Consensus of known TRAF-2 binding motifs
TVAVEETIPSF

SEQ ID NO: 16 - protein, 11 aa, Unknown
  Consensus of known TRAF-2 binding motifs
RTPIQEEQADA

SEQ ID NO: 17 - protein, 11 aa, Unknown
  Consensus of known TRAF-2 binding motifs
TGAAQEEDACS

SEQ ID NO: 18 - protein, 11 aa, Unknown
  Consensus of known TRAF-2 binding motifs
RCPQEEEGGGG

SEQ ID NO: 19 - protein, 11 aa, Unknown
  Consensus of known TRAF-2 binding motifs
VQTTQEEDGCS

SEQ ID NO: 20 - protein, 11 aa, Unknown
  Consensus of known TRAF-2 binding motifs
RFPEEEEGGCE

SEQ ID NO: 21 - protein, 11 aa, Unknown
  Consensus of known TRAF-2 binding motifs
RTPVQESGYPD

SEQ ID NO: 22 - protein, 11 aa, Unknown
  Consensus of known TRAF-2 binding motifs
RPPVQETGGGG

SEQ ID NO: 23 - protein, 11 aa, Unknown
  Consensus of known TRAF-2 binding motifs
HPPVQETGGGG

SEQ ID NO: 24 - protein, 11 aa, Unknown
  Consensus of known TRAF-2 binding motifs
HPPVQETGEGG

SEQ ID NO: 25 - protein, 11 aa, Unknown
  Consensus of known TRAF-2 binding motifs
HPPIQETGNGG

SEQ ID NO: 26 - protein, 11 aa, Unknown
  Consensus of known TRAF-2 binding motifs
SVPIQCTDKTD

SEQ ID NO: 27 - protein, 11 aa, Unknown
  Consensus of known TRAF-2 binding motifs
PHPQQATDDSS

SEQ ID NO: 28 - protein, 11 aa, Unknown
  Consensus of known TRAF-2 binding motifs
PYPIQATDGGN

SEQ ID NO: 29 - protein, 11 aa, Unknown
  Consensus of known TRAF-2 binding motifs
PHPIQATDGAN

SEQ ID NO: 30 - protein, 11 aa, Unknown
  Consensus of known TRAF-2 binding motifs
PHPVQASDGGD

SEQ ID NO: 31 - DNA, 22 bp, Artificial Sequence
  Mutagenic oligonucleotide designed to generate the TB1-SphK mutant
tgccactggcggcgccagtgcc

SEQ ID NO: 32 - DNA, 22 bp, Artificial Sequence
  Mutagenic oligonucleotide designed to generate the TB2-SphK mutant
caccgccagcggcgcccttaga

SEQ ID NO: 33 - DNA, 34 bp, Artificial Sequence
  Oligonucleotide used as a probe.
ggatgccattggggatttcctctttactggatgt